(19) United States
(12) Patent Application Publication
(10) Pub. No.: US 2010/0298744 A1
(43) Pub. Date: Nov. 25, 2010

(54) SYSTEM AND METHOD OF TREATING TISSUE WITH ULTRASOUND ENERGY

(75) Inventors: Gregory B. Altshuler, Lincoln, MA (US); Christopher Gaal, Mansfield, MA (US); Pavel Kamaev, North Reading, MA (US); Oldrich M. Laznicka, JR., Wellesley, MA (US); Ilya Yaroslavsky, North Andover, MA (US)

Correspondence Address: PALOMAR MEDICAL TECHNOLOGIES;NUTTER, MCCLENNEN & FISH LLP, SEAPORT WEST, 155 SEAPORT BOULEVARD, BOSTON, MA, 02210, US

(73) Assignee: PALOMAR MEDICAL TECHNOLOGIES, INC., Burlington, MA (US)

(21) Appl. No.: 12/771,558

(22) Filed: Apr. 30, 2010

Related U.S. Application Data
(60) Provisional application No. 61/174,201, filed on Apr. 30, 2009.

(51) Int. Cl. A61N 7/02

(52) U.S. Cl. 601/3

(57) ABSTRACT

Disclosed herein are method(s) and device(s) capable of generating a small lesion as deep as a few millimeters beneath the skin's surface and several cubic millimeters in volume in orders of magnitude less time, namely, tens of milliseconds. More specifically, in one exemplary embodiment, a method of treating tissue (e.g., skin) is provided which includes generating one or more ultrasound pulses with each pulse having a pulse width shorter than a thermal relaxation time of a tissue treatment volume, and applying one or more of said ultrasound pulses to at least one portion of the tissue treatment volume to generate one or more treatment areas in a region. Methods of treating tissue can include effecting a therapeutic treatment in said region of the tissue, and/or effecting a cosmetic treatment in said target region.

### CROSS-REFERENCE TO RELATED APPLICATIONS

This application claims the benefit of and priority to U.S. Provisional Application Ser. No. 61/174,201, which was filed on Apr. 30, 2009. This provisional application is herein incorporated in its entirety.

### FIELD OF THE INVENTION

The present disclosure relates to systems and methods for treating tissue with ultrasound energy.

### BACKGROUND OF THE INVENTION

Various devices and methods exist for generating high-intensity, focused shock waves using high intensity focused ultrasound. Most of the devices are experimental and only available in scientific laboratories. Methods employing high-intensity, focused shock waves are generally employed for ultrasonic induction of thermal lesions in cancerous tumors. Those methods involve prolonged sonication times to induce lesions in tissue. Such sonication times range from several seconds to several minutes or even dozens of minutes. In tumor treatment the area of treatment via sonication is generally small relative to the size of a patient's body.

### SUMMARY OF THE INVENTION

Employing currently available focused ultrasound technology in a less invasive manner and over a relatively large area of a subject's body would take a great deal of time with currently available sonication methods such as are employed in cancerous tumor treatment.

Disclosed herein are method(s) and device(s) capable of generating a small lesion as deep as a few millimeters beneath the skin's surface and covering several cubic millimeters in volume in orders of magnitude less time, for example, in tens of milliseconds. More specifically, in one exemplary embodiment, a method of treating tissue (e.g., skin) is provided which includes generating one or more ultrasound pulses with each pulse having a pulse width shorter than a thermal relaxation time of a tissue treatment volume, and applying one or more of said ultrasound pulses to at least one portion of the tissue treatment volume to generate one or more tissue treatment areas in a region. Methods of treating tissue can include effecting a therapeutic treatment in said region of the tissue, and/or effecting a cosmetic treatment in said target region.

Various ranges of pulse widths are provided herein. For example, the pulses can have a pulse width in a range of about 1 ms to about 70 ms, about 10 ms to about 50 ms, about 10 ms to about 40 ms, about 10 ms to about 30 ms, about 10 ms to about 20 ms, about 5 ms to about 10 ms, etc.

The pulses can also have any of a plurality of frequency ranges. For example, said pulses can have an ultrasound frequency in a range of about 0.7 MHz to about 20 MHz, 3.5 MHz to about 12 MHz, in a range of about 5 MHz to about 12 MHz, in a range of about 3 MHz to about 12 MHz, and/or in a range of about 20 kHz to about 700 kHz, etc.

The method can utilize various embodiments of a transducer configured to provide the desired ultrasound energy to the target tissue. For example, the method can include utilizing at least one transducer having a numerical aperture in a range of about 0.8 to about 1.1 to generate said one or more pulses such that each pulse exhibits a power density in a range of about 800 W/cm² to about 5000 W/cm², about 500 W/cm² to about 5,000 W/cm², and/or about 40 W/cm² to about 800 W/cm² at a focal area in the target region. For example, the method can include utilizing at least one transducer to generate said one or more pulses such that each pulse exhibits an energy density in a range of about 2.5 J/cm² to about 25 J/cm², and/or about 4 J/cm² to about 80 J/cm² at a focal area in the target region.

The method can also include targeting tissue at various locations and/or targeting various volumes and/or areas of tissue. For example, said target tissue region can be located at a depth in a range of about zero to about 6 mm below the tissue surface. Also, the target tissue region can have a volume in various ranges, for example, a volume in a range of about 1×10⁻⁴ mm³ to about 30 mm³.

The method can also include selecting various energy and/or treatment parameters so as to target the desired location. For example, the method can include selecting the ultrasound frequency of the pulses so as to cause damage and/or provide treatment in a tissue region extending from the tissue surface to a depth of about 6 mm below the tissue surface. The method can also include selecting the ultrasound frequency of the pulses so as to cause thermal action in the dermis layer of the tissue. The method can also include selecting the ultrasound frequency of the pulses so as to cause damage in a fatty region of the tissue. For example, this ultrasound frequency can be selected so as to be within a range of about 3 MHz to about 5 MHz, or about 3 MHz to about 12 MHz.

The method can also include focusing said ultrasound pulses into said target region. For example, the step of focusing the pulses can include focusing the pulses into a focal volume having a size in a range of about 0.0001 mm³ to about 30 mm³.

The step of applying ultrasound pulses to the tissue can include applying one or more pulses to each of a plurality of discrete tissue portions in said target region to generate a plurality of treated tissue portions separated from one another by untreated portions of said target region. For example, the treated portions can generate a plurality of separated coagulation lines within the target region. In another example, the treated portions can provide a pattern of separated treatment areas within the target region. Any of said treatment areas can be separated from a neighboring treatment area by any desired distance. For example, the distance between treatment areas can be in a range of about 1 mm to about 5 mm along at least one dimension.

The method can also include applying a substance to the tissue surface so as to enhance coupling of the ultrasound pulses into the tissue. For example, in one embodiment the substance is a gel.

In another aspect of the present disclosure, a method of applying ultrasound energy to the tissue is provided which includes applying at least one diagnostic ultrasound pulse to a tissue treatment volume within a target region, and detecting an echo generated in response to said diagnostic ultrasound pulse. The method can also include analyzing said echo to determine whether it is safe to apply ultrasound capable of generating treatment areas to said tissue treatment volume, and applying one or more ultrasound pulses each having a pulse duration shorter than the thermal relaxation time of the tissue treatment volume to the tissue treatment volume to cause one or more treatment areas therein. In one embodiment, the treatment areas effect a cosmetic treatment in the tissue target region. In another embodiment, the treatment areas effect a therapeutic treatment in the tissue target region.

The diagnostic pulse can be generated so as to have a frequency in any of a number of desired frequency ranges. For example, the frequency can be in a range of about 5 to about 15 MHz. Also, any of a variety of transducers can be configured to provide the desired energy delivery. For example, said diagnostic pulse can be generated by a transducer having a numerical aperture in a range of about 5 mm to about 20 mm. Also, the diagnostic pulse can be generated so as to exhibit any range of desired power density. For example, the power density can be in a range of about 0.1 to about 0.8 W/cm² at the tissue surface.

The method can also include a step or steps of analyzing said echo to determine the presence or absence of ultrasound obstacles along the propagation path of the diagnostic ultrasound pulse. For example, the method can include analyzing said echo to determine whether bone is present along the propagation path of the diagnostic ultrasound pulse, and/or analyzing said echo to determine whether sufficient coupling exists between the ultrasound pulse and the tissue.

In another embodiment, a method of treating tissue is provided which includes generating one of more ultrasound pulses each having a pulse width of less than about 95 ms. The method also includes applying one of more of said ultrasound pulses to at least one portion of a tissue target region to generate one or more treatment areas in said target region.

In another aspect, a method for applying ultrasound energy to tissue is provided which includes scanning an ultrasound transducer over a tissue surface (e.g., skin) to apply ultrasound energy to the tissue surface, and controlling the transducer so as to deliver the ultrasound energy to a plurality of tissue locations at a substantially uniform depth below the tissue surface. In one embodiment, the step of controlling the transducer includes causing said transducer to apply a substantially constant pressure to said tissue surface as the transducer delivers the ultrasound energy to said tissue locations. The step of controlling the distance can include maintaining a reference location of the transducer at a substantially constant distance relative to the tissue surface as the transducer delivers the ultrasound energy to said tissue locations. The step of scanning the transducer can also include activating the transducer to apply the ultrasound energy to selected locations of the tissue.

In one embodiment, the step of controlling the distance further includes causing the transducer to apply a substantially constant compressive pressure to the tissue surface while the transducer is delivering the ultrasound energy to each of said tissue locations. The method can also include the step of controlling the transducer to remove it from contact with the tissue surface upon termination of delivery of the ultrasound energy to at least one of said tissue locations. The method can include removing the compressive pressure upon termination of delivery of the ultrasound energy to at least one of the tissue locations. In one embodiment, the method can further include the step of raising the transducer relative to the tissue surface upon termination of delivery of the ultrasound energy to at least one of said tissue locations thereby facilitating the movement of the transducer from one treatment site to the next site. Also, the step of controlling the distance can further include causing the transducer to apply a substantially constant compressive pressure to the tissue surface while the transducer is delivering the ultrasound to said locations and raising the transducer a constant amount relative to the tissue surface while the transducer is moving over the tissue surface (e.g., from a one treatment site to another treatment site).

The method can also include the use of a transducer which is activated to apply one or more pulses of ultrasound energy to each of said tissue locations. Like above, various ranges of pulse width are provided. For example, said pulses can include a pulse width in a range of about 1 ms to about 70 ms, in a range of about 5 ms to about 20 ms, in a range of about 10 ms to about 50 ms, in a range of about 10 ms to about 40 ms, in a range of about 10 ms to about 30 ms, in a range of about 10 ms to about 20 ms, in a range of about 5 ms to about 10 ms, in a range of about 5 ms to about 70 ms, etc. Additionally, the method can utilize pulses having various ranges of frequency. For example, said pulse can have a frequency in a range of about 3.5 MHz to about 12 MHz, in a range of about 5 MHz to about 12 MHz, etc.

In yet another aspect, various embodiments of a device for applying ultrasound energy to tissue are provided. In one such embodiment, the device includes a transducer for generating ultrasound energy for application to the tissue. The device can also include a scanner coupled to the transducer for moving the transducer along three dimensions over tissue (e.g., skin) wherein the scanner can include a controller for controlling position of said transducer along a dimension substantially perpendicular to the tissue surface so as to deliver energy to a substantially uniform depth below the tissue surface at a plurality of tissue locations.

The controller can be configured to provide various ranges of motion. For example, the controller can be configured to cause said transducer to apply a substantially constant compressive force to the tissue as the transducer applies ultrasound energy to the tissue. The controller can also include a linear travel mechanism coupled to the transducer and configured to apply a substantially constant force to the transducer in a direction toward the tissue.

The linear travel mechanism can be configured in various manners so as to provide the desired application of force. For example, the linear travel mechanism can include a spring configured to effect application of said force. The linear travel mechanism can also include a pneumatic cylinder adapted to effect application of said force. The linear travel mechanism can also include a solenoid configured to effect application of said force.

In one embodiment, the controller can include a sensor for providing one or more signals indicative of a distance between a reference surface of the transducer and the tissue surface (e.g., skin surface). Various sensors can be utilized. For example, the sensor can be a force sensor, an optical sensor, or an electrical sensor (e.g., capacitance, e-field, inductive, resistive, etc.). The controller can also include a servo system in communication with said sensor wherein the servo system is configured to maintain a distance between said reference surface and the tissue surface at a predetermined value during said at least a portion of the scan based on signals provided by the sensor.

In yet another aspect, various embodiments of a device for applying ultrasound energy to tissue (e.g., a tissue surface, skin) are provided which include a transducer for generating ultrasound energy, and a scanner coupled to the transducer for moving the transducer relative to a reference position, the scanner can move in determined distances relative to the reference position, along at least two dimensions so as to deliver the ultrasound energy to selected locations of the tissue. For example, the scanner can use a reference position to move with known or determined distances relative to those reference positions. The reference position can be, for example, a coordinate relative to the device itself, or a feature (e.g., a spot or wrinkle) on the patient's body. That is, the scanner can be configured to track the transducer relative to its own coordinates or the skin.

In one embodiment, the scanner is configured to control movement of the transducer so as to deliver said ultrasound energy to each of said locations of the tissue a pre-defined number of times during a scan. The scanner can also be configured to control movement of the transducer so as to deliver said ultrasound energy to each of said locations once during a scan. In one embodiment, the scanner can be configured to track said transducer relative to one or more reference locations for controlling delivery of the ultrasound energy to said tissue locations.

In one embodiment, at least two dimensions are substantially parallel to the tissue surface and the scanner is configured to move the transducer along another dimension substantially perpendicular to the tissue surface. The scanner can also be configured to control movement of the transducer along the dimension perpendicular to the tissue surface such that the transducer delivers the ultrasound energy to said locations at a substantially uniform depth below the tissue surface.

In one embodiment, the device can further include a frame to which the transducer is coupled. The frame can be configured for contact with the tissue surface (e.g., skin) at one end thereof, wherein the end includes an opening through which the ultrasound energy generated by the transducer can be applied to the tissue. The frame can also be configured for immobilizing a portion of the tissue facing said opening of the frame when said end of the frame is in contact with the tissue. For example, the frame can be configured to stretch a portion of the tissue facing said opening of the frame when said end of the frame is in contact with the tissue.

In one embodiment, a method of treating tissue is provided which includes determining from an external surface of a skin a location of cellulite, and applying at least one diagnostic technique to a skin portion within the location of cellulite to determine a location of a connective strand. The method also includes applying one or more ultrasound pulses to the connective tissue strand, the one or more pulses having a pulse duration shorter than a thermal relaxation time of a target region treatment volume. As detailed below, a projection of connective tissue strand causes the appearance of cellulite.

Various diagnostic techniques are within the spirit and scope of the present disclosure. For example, the diagnostic technique can be diagnostic ultrasound, palpation, etc.

In yet another embodiment, a device is provided for applying ultrasound energy to tissue. The device includes a phased array of transducers for generating ultrasound energy, a controller coupled to the array wherein the controller is configured to target selected locations of a tissue treatment volume with ultrasound energy, and also configured so as to apply one or more ultrasound pulses each having a pulse width shorter than a thermal relaxation time of a tissue treatment volume.

In one aspect, a method for treating tissue includes applying to a region of tissue (e.g., in the volume of tissue) one or more heating acoustic pulses with a frequency of from about 0.7 MHz to about 20 MHz and with a power density and an energy density sufficient to raise the temperature in the region of tissue by at least about 5° C. The method for treating tissue also includes applying to the region of tissue one or more cavitation acoustic pulse with a frequency range of from about 20 kHz to about 700 kHz with a power density and an energy density sufficient to induce cavitation in the region of tissue (e.g., in the volume of tissue). In one embodiment, the one or more heating acoustic pulse has a frequency, a power density and an energy density sufficient to raise the temperature in the region of tissue from about 5° C. to about 35° C. The heating acoustic pulse can have a power density of from about 500 W/cm² to about 5,000 W/cm² and have an energy density of from about 2.5 J/cm² to about 25 J/cm². The cavitation acoustic pulse can have a power density of from about 40 W/cm² to about 800 W/cm² and have an energy density of from about 4 J/cm² to about 80 J/cm². Optionally, the cavitation acoustic pulse can be applied only after the heating acoustic pulse, in this way the region of tissue is heated by the heating acoustic pulse prior to treatment with the cavitation acoustic pulse. In another embodiment, the cavitation acoustic pulse is partially overlapped with the heating acoustic pulse and the delay between the cavitation acoustic pulse and the heating acoustic pulse ranges between about 1% and about 90% of the duration of the heating acoustic pulse.

In another aspect, a device for applying ultrasound energy to tissue includes an ultrasound transducer for generating ultrasound energy for application to tissue and a mechanism for dispensing at least one acoustic coupling medium between the ultrasound transducer and a tissue portion to provide a substantially constant acoustic coupling of the ultrasound energy into said tissue portion during treatment. In some embodiments, the mechanism is configured to continuously replenish the coupling medium during treatment. In other embodiments, the coupling medium is selected such that the speed of sound through the coupling medium is substantially the same as the speed of sound through the tissue. The ultrasound transducer can be disposed within a housing having a distal tip, the tip being sized and configured to dispense a desired amount of said at least one coupling medium onto a surface of said tissue portion (e.g., the surface of the skin). The at least one acoustic coupling medium can be a first coupling medium disposed within the housing. The device can include a reservoir containing a second coupling medium, the reservoir being in communication with the tip to transfer the second coupling medium to the tip to be dispensed onto said surface of the tissue portion.

The first coupling medium and the second coupling medium can be the same coupling medium. The first coupling medium can be a substance with an acoustic impedance substantially similar to an acoustic impedance of water. The first coupling medium can have a viscosity of up to about 500,000 cPs.

In some embodiments, the device further includes a drive mechanism in communication with the reservoir, the drive mechanism configured to drive a desired amount of the second coupling medium from the reservoir to the tissue surface. The drive mechanism may be, for example, a piston. Optionally, the drive mechanism and a viscosity of the second coupling medium are configured to dispense a desired amount of the second coupling medium at a desired rate so as to provide a thin film of the second coupling medium between the tissue surface and tip. In one embodiment, the thin film has a viscosity in a range of about 80,000 cPs to about 100,000 cPs. The thin film can: reduce friction between the tip and the tissue surface, provide cooling of the tissue surface, include an additive configured to provide an indication of prior treatment (e.g., the additive can be configured to change color if subjected to a predetermined amount of ultrasound energy). In one embodiment, the mechanism for dispensing at least one acoustic coupling medium is configured to dispense the coupling medium so as to substantially eliminate imperfections in the acoustic coupling medium during treatment.

In another aspect, a device includes a handheld housing, a source for generating ultrasound energy for application to tissue through a distal end of the housing, a reservoir for containing an acoustic coupling medium, the reservoir having an opening in proximity of the distal tip for dispensing the acoustic coupling medium onto a surface of the tissue to facilitate coupling of the ultrasound energy into the tissue portion. The device also includes a drive mechanism coupled to the reservoir for driving the acoustic coupling medium from the reservoir onto said tissue surface. In some embodiments, the drive mechanism and a viscosity of the acoustic medium are configured to provide a thin film of the acoustic medium onto the tissue surface as the housing is moved over the surface to apply ultrasound energy to said tissue.

In another aspect, a method of applying ultrasound energy to tissue includes providing a device having an ultrasound energy emitter and a reservoir for containing an acoustic coupling medium, the reservoir having an opening for dispensing said medium onto a tissue surface. The method includes moving the device over a tissue surface while dispensing the coupling medium from the reservoir so as to form a thin film of the coupling medium on the tissue surface and activating the emitter to apply ultrasound energy to the tissue surface having said film of the coupling medium. The film of the coupling medium facilitates coupling of the ultrasound energy onto the tissue. In one embodiment, the film has a substantially uniform thickness thereby to provide a substantially constant coupling between the ultrasound energy and different portions of the tissue. In another embodiment, the steps of dispensing the coupling medium and applying the ultrasound energy are performed substantially concurrently.

### BRIEF DESCRIPTION OF THE DRAWINGS

The present disclosure will be more fully understood from the following detailed description taken in conjunction with the accompanying drawings, in which:

FIG. 1 is a flow chart of potential steps of an exemplary embodiment of a presently disclosed method;

FIG. 2 is a schematic representation of an exemplary embodiment of a presently disclosed device;

FIG. 3 is an exploded view of an exemplary embodiment of a transducer assembly of a presently disclosed device;

FIG. 4 is a schematic representation of an embodiment of a system for determining tissue parameters during treatment;

FIG. 5A is a graph representing tissue temperature as a function of treatment time;

FIG. 5B is another graph representing tissue temperature as a function of treatment time;

FIG. 6 is a cross-sectional view of an exemplary embodiment of a transducer assembly of a presently disclosed device;

FIG. 7A is a top view of a volume of tissue of a treatment area with a plurality of target sites disposed therein in a grid-like pattern;

FIG. 7B is a side view of the treatment area shown in FIG. 7A;

FIG. 8 is a schematic representation of an exemplary embodiment of a device and a system;

FIG. 9 is a perspective view of an exemplary embodiment of a device;

FIG. 10A is a representation of a transducer assembly exerting a compressive pressure to a tissue surface above a target site;

FIG. 10B is a representation of removal of the transducer assembly of FIG. 10A from contact with the tissue surface thereby controlling the transducer to remove it from contact with a tissue surface above a target site;

FIG. 10C is a representation of the transducer assembly moving laterally from the first target site towards a position above a second target site;

FIG. 10D is a representation of the transducer of FIG. 10A exerting another compressive pressure to the tissue surface above the second target site;

FIG. 11 is a schematic representation of a feedback mechanism of an exemplary embodiment of a presently disclosed system;

FIG. 12 is a view of an exemplary embodiment of a transducer assembly;

FIG. 13A is a view of a transducer assembly;

FIG. 13B is a view of a transducer assembly;

FIG. 14A is a schematic representation of a phased array of transducer elements;

FIG. 14B shows an embodiment of a transducer that combines two separate transducers;

FIG. 14C shows a cross section of embodiment of a transducer that combines two separate transducers shown in FIG. 14B;

FIG. 14D shows an embodiment of another transducer that combines two separate transducers;

FIG. 15 is an image of pig skin tissue treated with ultrasound energy;

FIG. 16 is a representation showing a target site located at a depth below the tissue surface;

FIG. 17 is an image of skin tissue treated with ultrasound energy;

FIG. 18 is a graph of time of ultrasound impulse versus focal plane beneath a tissue surface;

FIG. 19 is an image of human skin tissue treated with ultrasound energy;

FIG. 20 is another image of human skin tissue treated with ultrasound energy;

FIG. 21 is another image of pig dermis tissue treated with ultrasound energy;

FIG. 22 provides twelve images of pig skin tissue treated with ultrasound energy, with each image of a different depth relative to the surface of the pig skin tissue;

FIG. 23 is an image of a sagittal section of swine tissue treated with ultrasound energy;

FIG. 24A is an image of a cross-section of “healthy” tissue that does not have the appearance of cellulite; and

FIG. 24B is an image of a cross-section of “cellulite” tissue that has the appearance of cellulite.

### DETAILED DESCRIPTION

Certain exemplary embodiments will now be described to provide an overall understanding of the principles of the structure, function, manufacture, and use of the systems, devices, and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present disclosure is defined solely by the claims. The features illustrated or described in connection with one exemplary embodiment may be combined with the features of other embodiments. Such modifications and variations are intended to be included within the scope of the present disclosure.

Methods, devices, and systems for applying ultrasound energy to tissue, e.g., skin tissue, are disclosed. In some cases, the ultrasound energy can be applied as a plurality of ultrasound pulses to selected tissue locations, e.g., skin tissue at a depth below the skin surface. In some embodiments, the pulse widths are selected to be less than about 70 milliseconds (ms), e.g., in a range of about 0.1 ms to about 70 ms, and/or in a range of about 5 ms to about 50 ms. Further, in some embodiments in which the ultrasound energy is applied to the tissue surface (e.g., to skin tissue), one or more transducers generating the ultrasound energy can be controlled so as to deliver the energy to a substantially uniform depth below the tissue surface (e.g., skin surface). In some cases, the ultrasound energy can be applied via a disclosed device while the device is scanned over the skin surface. To facilitate the scanning of the device over the skin, a suitable substance, e.g., a gel can be dispensed onto the tissue surface, e.g., in a substantially continuous manner and/or at a substantially consistent thickness on the tissue surface. In one embodiment, the gel is dispensed onto the tissue surface from a container attached to the device. The supply of gel onto the tissue surface can lower the friction between at least a portion of the device (e.g., the device tip) and the tissue surface (e.g., the skin). In addition, employing such a gel substance on the surface of the tissue being treated can facilitate the delivery of the ultrasound energy to the tissue by providing acoustic impedance matching.

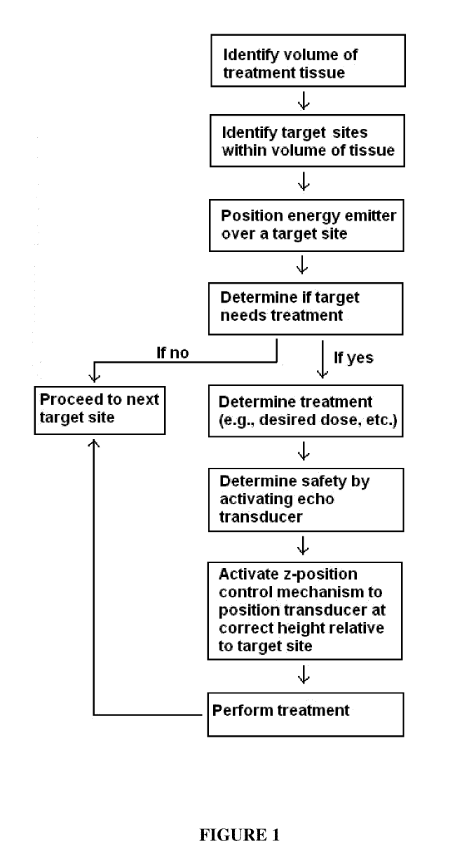

FIG. 1 provides an overview of steps in an exemplary method. That is, the method can include identifying a volume or area of tissue to be treated, and identifying any number of target sites within the volume or area of tissue, for example, the number of target sites can be identified by selecting a density or periodicity of treatment in the previously identified volume or area of treatment tissue. The method can include positioning an energy emitter (e.g., a transducer) at a position substantially over the target site, and determining if the target site has been treated before, if the target site is in need of treatment, if the site has been treated but needs additional treatment, etc. If the target site is not in need of treatment, the method can include a step of moving the energy emitter towards the next target site, e.g., “Proceed to Next Target Site”.

If the target site is in need of treatment, the method and system can determine the desired treatment (e.g., the desired treatment regimen). As detailed below, the desired treatment regimen can include dose, duration, etc. Prior to initiating treatment, the system and method can be configured to determine if it is safe and/or efficient to deliver energy to the target site. For example, the method can trigger an echo transducer configured to detect the presence or absence of bone, the effectiveness of coupling of the transducer to the tissue (whether the coupling is good or poor), etc. The method can also activate a z-position control mechanism configured to position the head of the energy emitter at a known z-position relative to the target site thereby allowing a known amount of energy to be delivered to a known depth below a tissue surface. Once the transducer is properly positioned, the emitter can be activated and the desired treatment performed. The transducer can then be moved to the next target site for further treatment.

With reference to the step of the determining treatment in the flowchart of FIG. 1, in an exemplary method for applying ultrasound energy to tissue (e.g., to a tissue surface such as skin), one or more ultrasound pulses are generated. Heating of tissue with ultrasound energy has previously been taught, however, prior ultrasound treatments employ relatively long pulse widths, to produce large heated zones. For example, U.S. Pat. No. 6,595,934 teaches a pulse width between 2 seconds and 60 seconds. Such long treatment times cause heat diffusion and risk an undesirable increase in damage volume. In accordance with the present disclosure, pulse widths shorter than the thermal relaxation time of the intended volume of thermal action τᵣ can be employed:

τᵣ=d²/4α

where:

d is characteristic dimension of the area, and

α is the thermal diffusivity (for water-rich tissue such as dermis, α≈1.5*10⁻³ cm²/s).

In one embodiment, if target dimension of the area d≈z 0.2 mm, then the thermal relaxation time is ˜70 ms. Using relatively short pulses as disclosed herein enables one or more of (1) confinement of the thermal energy within the intended treatment zone, (2) sharp demarcation of the boundaries of the thermal action zone, and (3) treatment with short pitch and high density of the thermal action zones.

In an exemplary embodiment, each pulse has a pulse width in a range of about 1 ms to about 70 ms, and the pulses are applied to at least one portion of a tissue target region to generate one or more treatment volumes in said region. By way of example, in some cases, the pulse widths can be in a range of about 10 ms to about 40 ms, or in a range of about 10 ms to about 30 ms, or in a range of about 10 ms to about 20 ms, or in a range of about 5 ms to about 10 ms, or in a range of about 0.1 ms to about 5 ms. As taught herein, the application of such short ultrasound pulses at proper power densities, such as those disclosed below, can effect a desired cosmetic and/or therapeutic treatment in the tissue while minimizing, and preferably eliminating, discomfort experienced by a subject during application of the pulses. The term “treatment” is used herein to encompass both a cosmetic and a therapeutic treatment. Examples of a cosmetic treatment can include, without limitation, skin rejuvenation (e.g., improving skin color, skin tightening, treatment of rhytides, treatment of wrinkles, etc.) and reduction and/or elimination of cellulite appearance. Examples of a therapeutic treatment can include, without limitation, treatment of tissue in order to illicit a response by which immunity is bolstered or strengthened. While not being bound to any single theory it is believed that therapeutic treatment can be achieved according to the disclosed methods, because treatment of the tissue with the ultrasound energy creates injury and/or stress to the treated tissue, which promotes inflammation and adaptive immunity that is capable of resisting and/or fighting threats to health. In addition, therapeutic treatments can result from an increase in blood supply to the treated area.

In some embodiments, the ultrasound pulses have a frequency in a range of about 3 MHz to about 12 MHz or in a range of about 5 MHz to about 10 MHz or a frequency of about 7 MHz. The frequency of the pulses can be selected based on any of a number of variable factors including, the desired cosmetic and/or therapeutic treatment, the location(s) of the tissue to be treated (e.g., the depth of the skin tissue below the skin surface, whether downtime associated with tissue treatment is desirably minimized and/or avoided), the type of tissue to be treated, among other factors. For example, ultrasound pulses with a frequency in a range of about 3 MHz to about 5 MHz can be employed for treating fatty tissue (e.g., by causing intentional regions of thermal damage in the tissue). Generally, for a set length treatment time (t), the higher the frequency the shallower the depth of penetration in the z direction into the tissue relative to a lower frequency, which results in a deeper depth of penetration in the z direction into the tissue in the same treatment time (t). For example, attenuation of ultrasound in tissue at 12 MHz is from about 5 to about 9 times higher than attenuation at 3 MHz depending on the nature of the tissue (e.g., if the tissue is muscle or fat, attenuation in skin being the largest, then muscle and fat).

In many embodiments, the ultrasound pulses are focused onto the tissue, e.g., into a focal volume having a volumetric size in a range of about 0.0001 mm³ to about 30 mm³, to provide a desired power density. By way of example, the power density of the applied pulses can be in a range of about 800 W/cm² to about 5000 W/cm² at the focal area, or a range of from about 1200 W/cm² to about 2000 W/cm². In some cases, the pulses are focused onto the tissue with a numerical aperture in a range of about 0.05 to about 0.9, in a range of about 0.5 to about 1.1, in a range of about 0.8 to about 1.1, or about 1.0 (e.g., an aperture of 1.0 in use with 5 MHz).

In the above ultrasound method, the ultrasound energy, e.g., in the form of a plurality of ultrasound pulses, can be applied to a variety of tissue types. In some embodiments, the ultrasound energy is applied to the skin to treat skin tissue, e.g., tissue at a depth in a range of zero to about 6 mm, or from about 2 mm to about 5 mm below the tissue surface (e.g., the skin surface). In some embodiments, the ultrasound energy (e.g., ultrasound pulses) can be applied to each of a plurality of discrete tissue portions in a target tissue region to generate a plurality of treated tissue portions separated from one another by untreated portions of the target region. By way of example, a treatment portion can be separated from a neighboring treatment portion by a distance in a range of about 1 mm to about 5 mm, or from about 2 mm to about 4 mm, or about 3 mm. In some cases, the treated portion provides a pattern of separated treatment regions within a tissue target region. By way of example, the treated portions can generate a plurality of separated coagulation lines within a target region (a region requiring treatment).

With reference to the flowchart of FIG. 1, in one embodiment of a method disclosed for applying ultrasound energy to the tissue, at least one diagnostic ultrasound pulse is applied to a tissue portion within a target tissue region, and an echo generated in response to said diagnostic ultrasound pulse is detected. The echo can be analyzed to determine whether it is safe to apply ultrasound energy capable of generating treatment regions to said tissue portion. If it is safe to apply the ultrasound energy, one or more ultrasound pulses each having a pulse duration in a range of about 5 ms to about 70 ms can be applied to that tissue portion to cause one or more treatment regions therein.

In some cases, the diagnostic pulse can have a frequency in a range of about 5 MHz to about 15 MHz and can exhibit a power density in a range of about 0.1 W/cm² to about 0.8 W/cm² at the tissue surface, or at a focal area when the diagnostic pulses is focused onto the tissue. The focal depth of the diagnostic pulse will range from about zero to about 6 mm, or from about 2 mm to about 5 mm below the tissue surface.

In some embodiments, the generated diagnostic echo can be analyzed to determine the presence or absence of any obstacles along the propagation path of the diagnostic ultrasound. An ultrasound obstacle can include, for example, bone and/or bubbles in a topical substance (e.g., a gel) applied to a tissue surface (e.g., skin surface) for facilitating coupling of the ultrasound energy into tissue and/or regions of inconsistent density in the applied topical substance. In some cases, the echo can be analyzed to determine whether sufficient coupling exists between the diagnostic ultrasound pulse and the tissue (e.g., by analyzing the intensity of the returning echo). The presence of bone within a depth of from about 2 mm to about 3 mm below the focus area risks thermal damage to the bone that can result in, for example, necrosis to the bone. Improper coupling due to, for example, bubbles or inconsistent gel density, can result in inconsistent energy delivery to the focal area resulting in inconsistency or absence of treatment to the focal area in a region of a bubble and/or inconsistent gel density.

With reference to the flowchart of FIG. 1, in one exemplary embodiment of a method for applying ultrasound energy to tissue (e.g., skin tissue), an ultrasound transducer can be scanned over a tissue surface (e.g., skin surface) to apply ultrasound energy to the tissue. The transducer can be controlled so as to deliver the ultrasound energy to a plurality of tissue locations at a substantially uniform depth below the tissue surface (e.g., below the skin surface).

By way of example, in some cases, the transducer can be controlled to apply a substantially constant compressive pressure to the tissue surface (e.g., skin surface) as the transducer delivers the ultrasound energy to the tissue locations. Upon termination of the application of the ultrasound energy to a tissue location, the compressive pressure can be removed (such that a transducer is removed from contact with the tissue, while the transducer is moved to another location to which the ultrasound energy will be applied). As another example, a reference location (e.g., a radiation-emitting surface) of the transducer, or a reference location of a frame to which the transducer is coupled, can be maintained (e.g., via feedback control) at a substantially constant distance relative to the tissue surface (e.g., skin surface) as the transducer delivers the ultrasound energy to the tissue such that at each irradiation location, the energy is effectively deposited at a substantially uniform depth.

While the various embodiments provided herein describe the treatment of tissue located at some depth below the skin surface, various other such tissues may be treated. Those skilled in the art will appreciate that various other types of tissue are within the spirit and scope of the present disclosure.

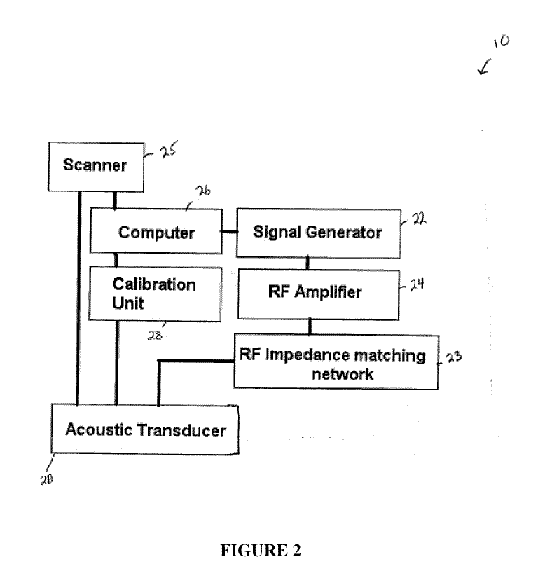

FIG. 2 is a block diagram of an ultrasound device 10 according to one embodiment that can be utilized to apply ultrasound energy to tissue, e.g., in a therapeutic noninvasive manner. The device 10 includes an acoustic transducer 20 for generating ultrasound energy, e.g., in the form of one or more ultrasound pulses. More specifically, a signal generator 22 produces an electrical signal having a desired frequency (e.g., a frequency in a range of about 3 MHz to about 12 MHz), which is amplified by an amplifier 24 (e.g., a RF Amplifier) and applied to the acoustic transducer 20 (e.g., a piezoelectric element), which transforms that electrical signal to an acoustic signal (wave) to be applied to tissue. The RF Impedance matching network 23 matches the signal from the RF Amplifier 24 with the Acoustic Transducer 20 to filter out any impedance mismatch. The computer 26 can selectively activate and deactivate the signal generator 22 to cause the generation of one or more acoustic pulses by the acoustic transducer 20. The acoustic transducer 20 can be coupled to an acoustic focusing element that can focus the acoustic pulses generated by the acoustic transducer 20 into the tissue.

The device 10 further includes a calibration unit 28 that can initially calibrate the transducer 20 and confirms that the transducer 20 is functional. The calibration unit 28 is in communication with the computer 26 to provide information regarding the status of the acoustic transducer 20. In some cases, the computer 26 can utilize this information to selectively activate the signal generator 22, and hence the acoustic transducer 20, to apply ultrasound pulse(s) to selective locations of the tissue. For example, in some cases, the computer 26 can compare the information received from the calibration unit 28 to a pre-defined pattern of locations to determine when the transducer 20 needs to be activated. The computer 26 communicates with the scanner 25 to direct the scanner 25 to move the transducer 20 in a desired pattern.

The acoustic transducer 20 can scan over the skin surface and emit focused ultrasound waves to deliver acoustic energy into a plurality of tissue locations at a precise depth in the subcutaneous tissue. In some embodiments, the scanning procedure can include movement of the transducer 20 over the tissue surface with deceleration of the transducer 20 speed at target treatment spots. One or more ultrasound pulses can be fired to treat the treatment spot. After the firing of the ultrasound pulse(s), the transducer 20 can move, e.g., at a predetermined acceleration, to an adjacent target treatment spot to apply ultrasound pulse(s) to that spot.

In some embodiments, the scanning procedure can include continuous movement of the transducer 20 over the tissue surface with energy delivered in a continuous manner, e.g., during at least a portion of the scan. By way of example, the ultrasound energy and the speed of the transducer 20 can be selected such that the continuous movement of the transducer 20 delivers energy in a continuous manner to create a line of coagulation in the tissue without damaging the surface of the tissue (e.g., the skin surface).

In some embodiments, the transducer 20 can receive an electrical energy up to about 30 Watts from the signal generator 22 and the amplifier 24 to generate acoustic power in a range of about 15 Watts to about 20 Watts for application to the skin. As noted above, in many embodiments, the acoustic energy generated by the transducer 20 can be focused into the tissue to provide a power density in a range of about 800 W/cm² to about 1500 W/cm² at a focal area of the target region that ranges from about zero to about 6 mm below the tissue surface.

In some implementations, the above exemplary device can operate in two modes: one modality in which predominantly mechanical (non-thermal) energy is used to disrupt fat cells and another modality in which higher frequency ultrasound energy is used to cause thermal action by some increase in temperature in targeted volumes (e.g., small volumes) of subcutaneous connective tissue.

The device 10 can be implemented to generate one or more desired ultrasound frequencies, e.g., in a range of about 3 MHz to about 12 MHz. As the applied ultrasound frequency increases, the length of the focal zone and the volume of tissue coagulation caused by the focused ultrasound decreases. As discussed further below, a prototype device built in accordance with the teachings herein was utilized to apply ultrasound energy to skin tissue at any of a number of frequencies including, for example, about 3.5 MHz, about 5 MHz, and about 12 MHz. The frequency that is applied depends on, for example, the desired size of coagulation. In one embodiment, at a frequency of about 12 MHz, at a time of about 60 ms, relatively small and relatively superficial damage is produced in skin beginning from the surface to a depth of about 1 mm with a maximum diameter of 0.5 mm at the surface of the skin. A frequency of about 5 MHz, at a time of from about 10 ms to about 20 ms, was chosen to produce relatively small (e.g., between about 0.1 mm and about 1 mm in size) damage in the dermis at a depth of from about 1 mm to about 3 mm. A frequency of about 3.5 MHz was chosen to produce relatively large (e.g., about 2 mm in length and about 0.5 mm in diameter) damage in predominantly fatty region during 110 ms under the skin at 7 mm depth.

As noted above, in many embodiments, the ultrasound energy (e.g., the ultrasound pulses) can be focused into tissue. The F-number is a measure of how rapidly the ultrasound energy is focused into the tissue, and is defined as the ratio of the focal length relative to the transducer's active aperture diameter. In general, as the focusing of the ultrasound energy becomes stronger (as the F-number decreases), the smaller is the near-field heating produced by the transducer. In some embodiments, the F-number associated with the focusing of the ultrasound generated by the transducer 10 into the tissue can be, e.g., in a range of about 0.8 to about 1.1. By way of example, in some embodiments, an F-number of about 1 (or smaller) can be utilized to minimize near-field heating that can result from a series of sound impulses. Increasing the frequency of the pulses and/or the delay between successive pulses, as well as decreasing the pulse duration and F-number of the transducer 10, can reduce near-field heating during operation.

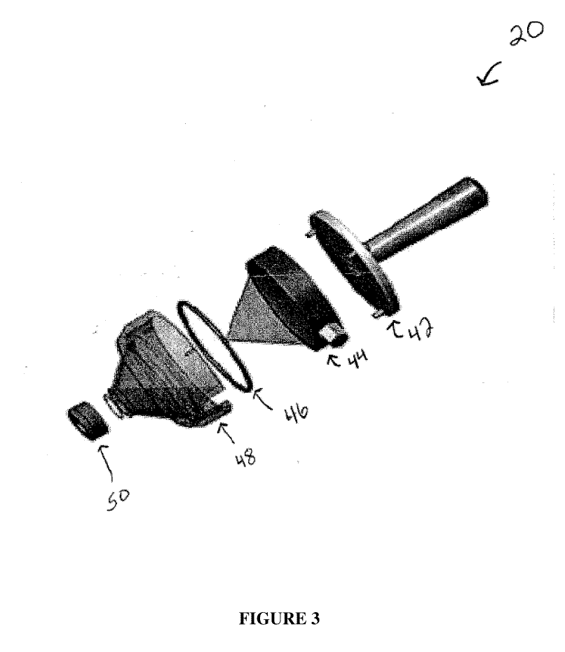

FIG. 3 schematically depicts a focusing acoustic transducer assembly 20 in accordance with one embodiment that can generate and focus ultrasound energy (e.g., high intensity ultrasound pulses) onto the tissue. The exemplary transducer assembly 20 includes a transducer element 44 for generating acoustic waves that is coupled to a handle 42. The transducer element 44 is coupled to a plastic coupling cone 48. A rubber ring 46 provides a seal between the transducer element 44 and the coupling cone 48. A removable and replaceable tip 50 can be attached to an end of the cone 48. The transducer element 44 provides a predetermined focal point that is based upon the curvature of the transducer. The transducer assembly 20 includes a cone 48 and a tip 50 that combine to contact the patient's tissue surface. Because the transducer element 44 provides a predetermined focal point, the distance that the combined cone 48 and tip 50 create relative to the tissue surface is determinative of the focal depth achieved in the tissue being treated. Accordingly, one or more of the cone 48 and tip 50 may be interchanged to achieve a desired focal depth.

In one embodiment, when attached to the cone 48 one side of the tip 50 contacts the patient's tissue surface. The thickness of the tip 50 will determine in part and in combination with the cone 48, how deep into the patient's tissue the focal depth penetrates. A less thick tip 50 will provide a deeper focal depth than a thicker tip, which will provide a less deep or more shallow focal depth. Suitable tips 50 can range in size (e.g., thickness) to achieve depths ranging from zero to 6 mm below the tissue surface. Likewise, suitable cones 48 can be sized to achieve in combination with a tip 50 depths ranging from zero to about 6 mm below the tissue surface. Suitable cones 48 and/or suitable tips 50 may be made from machined metal, a polymer, e.g., a low cost plastic suited to disposability. The cone 48 may be made from the same or different material than the tip 50 to which it mates.

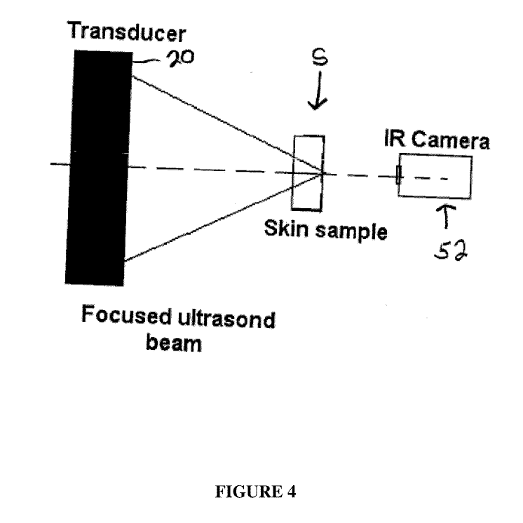

The ultrasound energy that is concentrated in a focal spot (region) can temporarily increase the tissue temperature and cause coagulation of the tissue. In some embodiments, the transducer 20 as well as the focusing element (herein also referred to as a focusing lens) can be configured to create treatment regions (e.g., coagulated tissue regions) with dimensions that can range from about several hundred micrometers to a few millimeters. In one illustrative exemplary study, samples of tissue were sonicated for about twenty milliseconds with focused ultrasound at a frequency of about 3.5 MHz and at an acoustic power density of up to 900 W/cm² at the focal peak. The ultrasound energy was focused into the tissue at an F-number of about 0.9. An infrared (IR) thermal camera was utilized to measure temperature rise in the tissue due to exposure to the focused ultrasound energy. More specifically, FIG. 4 schematically depicts the set-up utilized for measuring the temperature rise by the IR-thermal camera 52. The ultrasound energy generated by the transducer 20 was focused into a tissue sample (e.g., a skin sample) S, and an IR camera 52 was employed to record the temperature from the back of the sample S from about 3 mm and from about 5 mm below the tissue surface through which the ultrasound energy was focused into those depths below the tissue surface.

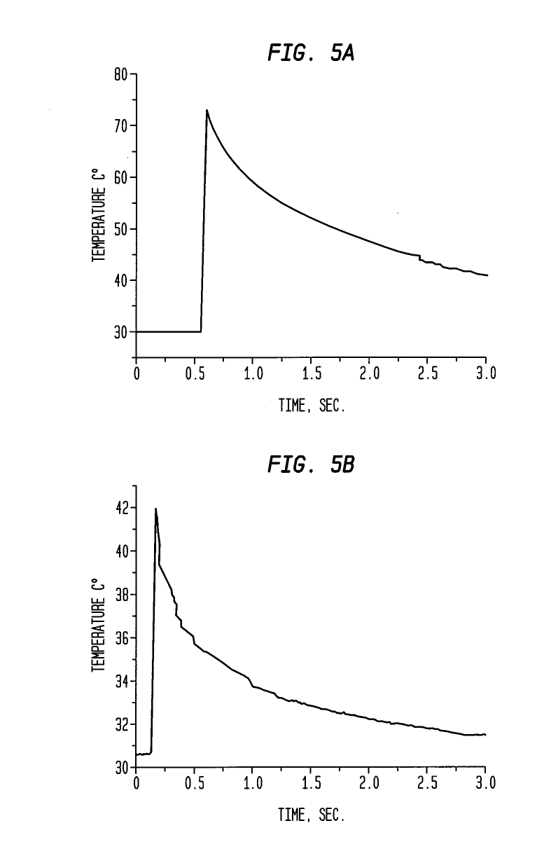

FIGS. 5A and 5B show temperature profiles of the skin region exposed to ultrasound energy for a period of about 20 milliseconds focused, respectively, at about 3 mm (FIG. 5A) and at about 5 mm (FIG. 5B) beneath the skin surface with a frequency of about 3.5 MHz. A camera recorded the temperature changes in the focal area during sonication. These temperature profiles show that short ultrasound pulses can be utilized to raise the tissue temperature to values needed for tissue coagulation.

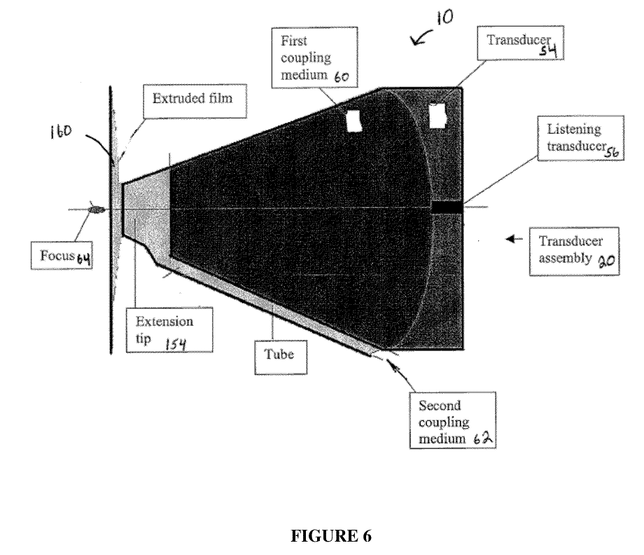

In some embodiments, referring to FIG. 6, an ultrasound device 10 can include two acoustic transducers, one of which is employed for generating ultrasound energy for treatment and the other for generating ultrasound energy for imaging/diagnostic purposes. By way of example, FIG. 6 schematically depicts a transducer assembly 20 that includes a large focused transducer 54 and a smaller transducer 56 (e.g., a listening transducer), which is placed in a central opening of the large transducer 54. The ultrasound energy generated by the transducers 54, 56 passes through coupling media 60 and 62 (and optionally through a topical substance applied to the skin surface) to be delivered to the tissue surface to treat a tissue region, e.g., to generate a coagulation region 64 in the focusing region below the tissue surface.

The small transducer 56 is a listening transducer that is capable of receiving a returning echo generated in response to a diagnostic acoustic wave generated by the listening transducer. By way of example, such an echo can be created by the reflection of the acoustic wave from (a) a discontinuity in a topical substance (e.g., gel) applied to the tissue surface to facilitate coupling of the acoustic wave to the tissue, (b) surface of the tissue, (c) bone or other solid object(s) which could be below the tissue surface. The retuning echo can be measured to determine whether at least an object that would interfere with treatment acoustic wave is present in the path of the waves, and if so, to determine how far away the object is, its size, shape, consistency (e.g., whether the object is solid, filled with fluid, or both), and uniformity. Such information can be gleaned, e.g., based on the amplitude (strength), frequency of the echo and the time it takes for the echo to be detected relative to the transmission of the diagnostic acoustic wave. Such analysis of the echo can be performed by a computer (such as a computer 26 shown in the above device 10 in FIG. 2) that receives electrical signal(s) generated by the diagnostic transducer 20 in response to the detection of the echo.

In practice, one or more diagnostic acoustic pulses are applied to the tissue prior to the application of treatment acoustic energy. If the analysis of one or more echo signals detected in response to such diagnostic pulses indicates that no interfering objects (or conditions such as discontinuity in a topical substance applied to the skin surface) are present, the treatment transducer 54 can be activated to generate treatment acoustic energy (e.g., in the form of pulses) for application to the tissue.

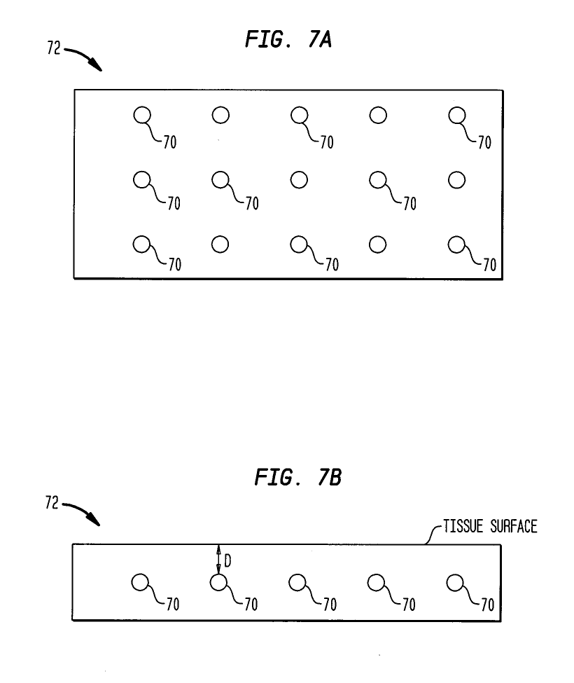

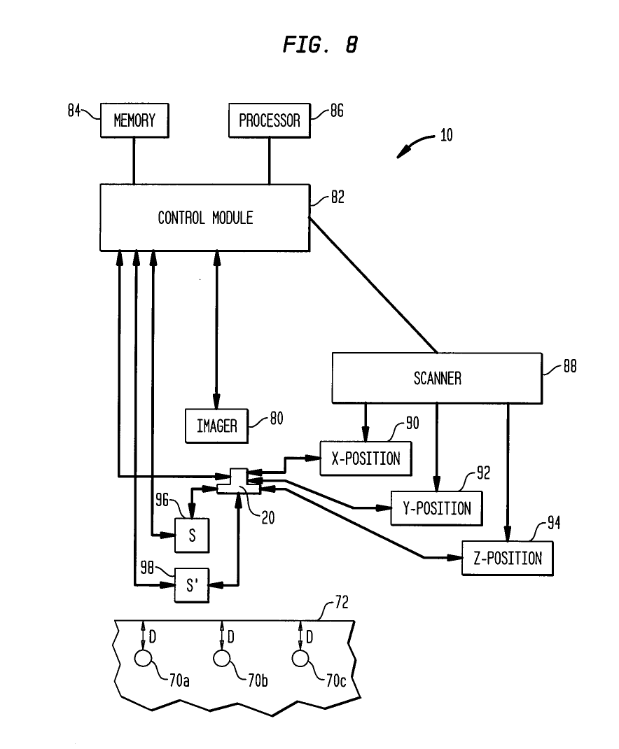

In some embodiments, ultrasound energy can be applied to selected locations of a tissue region requiring treatment to generate treatment portions that are separated from untreated portions within that region. The untreated portions can facilitate the healing process. By way of example, as shown schematically in FIGS. 7A and 7B, providing top and side views of a volume of treated tissue, a pattern (or a random collection) of separate coagulated tissue portions 70 can be formed, via application of ultrasound pulses, in a region 72 of tissue requiring treatment. As discussed in more detail below, such treatment of the skin tissue (or other tissue), which is herein also referred to as fractional treatment, can be achieved by scanning a transducer 20 over the tissue surface and selectively activating the transducer 20 to apply acoustic energy to specific, target locations 70 of the region of tissue 72.

In another embodiment, methods, devices, and systems for controlling the movement and positioning of the transducer 20 (or other energy source) relative to a tissue surface (e.g., skin) allow accurate delivery of a pre-determined amount of energy to specific target tissue sites (e.g., target sites 70 in FIGS. 7A and 7B). In an exemplary embodiment, these target tissue sites are located at some depth (e.g., “D” in FIG. 7B) below a tissue surface (e.g., skin surface), thereby requiring the device 10 to account for this depth in providing the specific amount and/or type of energy necessary to specifically target those sites. In some cases, the device 10 includes a mechanism for controlling the transducer 20 so as to deliver energy to tissue locations disposed at a substantially uniform depth below the tissue surface. Additionally, some treatments require that a constant amount of energy be delivered to each site. For such treatments, in some embodiments, the device 10 is capable of accounting for any differences and/or irregularities in the tissue areas above each of the various target sites so as to deliver a constant energy dose to those sites. Some exemplary devices and systems that account for such differences and/or irregularities are described in connection with FIG. 6. For example, FIG. 6 shows a listening transducer 56 that can be employed in conjunction with the transducer 54. The listening transducer 56 receives reflected echo from the spot treated with the transducer 54. When the transducer 54 focuses energy at location 64 an ultrasonic echo reflects back to the listening transducer 56 so that based on the information received by the listening transducer 56 irregularities in the tissue areas can be detected and the level of energy applied to the transducer 54 can be adjusted to compensate for the irregularities.

FIG. 8 provides another overview of components of an exemplary device and system 10. In some implementations, the device 10 can include an imager or sensing device 80 configured to record a tissue image that can be utilized to define a tissue area over which an energy source (e.g., a transducer) can be scanned to apply energy to the tissue and/or to provide information regarding tissue locations requiring treatment. The imager 80 can be in communication with a control module 82 having at least a memory 84 for storing various forms and types of data, and a processor 86 for interpreting the data, making calculations, implementing any number of stored algorithms, etc. In an exemplary embodiment, the control module 82 can identify one or more tissue target sites to which energy (e.g., ultrasound energy) should be applied. In various treatment procedures, these target sites can be located at some distance D (see FIGS. 7B and 8) below a tissue surface (e.g., skin surface). As detailed below, the control module 82 can utilize stored algorithm(s) to select tissue target sites. Such sites can be selected based on some analysis of the recorded image(s) of the treatment area, and/or the sites can be selected by various other mechanisms.

In this exemplary embodiment, the control module 82 is also in communication with a scanner or scanning mechanism 88. The scanning mechanism 88 is, in turn, in communication with an ultrasound energy emitter (e.g., a transducer) 20. Based on instructions from the control module 82, the scanning mechanism 88 can position the energy emitter 20 above a target site via an x-position control mechanism 90 and/or a y-position control mechanism 92. In many embodiments, the scanning mechanism 88 can also move the transducer 20 along the z direction (typically a direction that is substantially perpendicular to the skin surface) via a z-position control mechanism 94. In some embodiments, the scanning mechanism 88, again in combination with the control module 82, can also determine the height that the transducer 20 should be positioned above the target site so as to ensure that a desired amount of ultrasound energy will be delivered to the target site, and position the energy emitter 20 at this determined height. As detailed below, a sensor 96 in communication with a z-position control mechanism 94, and in further communication with the control module 82, can determine the proper height and move the energy emitter 20 accordingly.

Once the transducer 20 is positioned above a target site, the control module 82 can actuate the energy emitter 20 so as to deliver a desired amount and type of ultrasound energy to the target site for a desired amount of time. The scanner 88 can then move the transducer 20 to position it over another target site to apply ultrasound energy to that site. In some embodiments, the device 10 can also include various additional sensors 98 configured to sense one or more parameters of the tissue surface and/or target site so as to determine if treatment should be stopped in view of some safety concern and/or the effect of the treatment. This information can be transferred to the control module 82 which can then determine based on, for example, some stored treatment protocol and/or algorithm, whether this site should be targeted again, and if so, whether such further treatment should be performed immediately or at some future time point (e.g., the energy emitter can be returned to this initial site after the remaining target sites have been treated). Thus, these features provide the device 10 with a feedback mechanism configured move the energy emitter 20 among the various target sites until each site has been treated once or repeatedly, as needed.

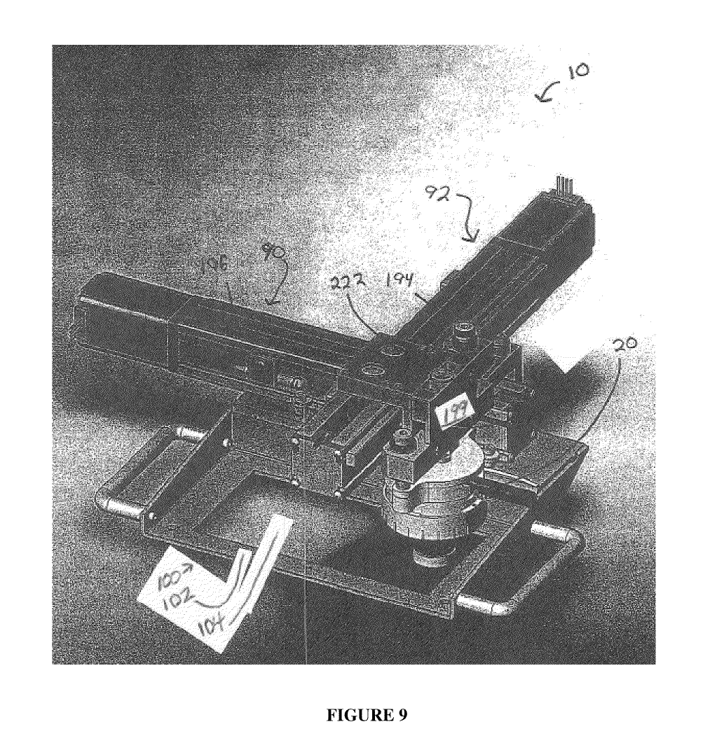

FIG. 9 provides an exemplary implementation of a device 10. In general, the device 10 includes an energy emitter 20 in communication with a scanning mechanism 88 (shown schematically in FIG. 8). The energy emitter 20 can be any emitter capable of delivering a desired amount and type of ultrasound energy to a target site. In an exemplary embodiment, the energy emitter is a piezoelectric transducer. An exemplary embodiment of the transducer assembly is described above and shown in FIG. 3.

The energy emitter 20 can be coupled to a scanner or scanning mechanism 88 which can move the energy emitter 20 to some desired position relative to the target site(s). The scanning mechanism 88 can be configured to achieve the desired x-, y-, and/or z-position relative to a target tissue site. For example, the scanning mechanism can be in communication with various sensors 96, 98 and/or a control module 82 so as to automatically move the energy emitter 20 from one desired position to the next desired position. Alternatively, the scanning mechanism 88 can be manually manipulated from one desired position to the next desired position. Also, the scanning mechanism 88 can be controlled by some combination of automation and manual control.

In an exemplary embodiment, the scanning mechanism 88 can be configured to control movement of the transducer 20 relative to an x-axis and a y-axis thereby allowing the energy emitter 20 to be positioned in two dimensions substantially above a target site. The scanning mechanism 88 can also be configured to control the height (i.e., z-position control) of the energy emitter 20 relative to a tissue surface thereby ensuring a desired amount of energy is delivered to a desired depth below the tissue surface so as to effectively treat the target tissue.

FIG. 9 provides an exemplary implementation of the scanning mechanism. That is, the scanning mechanism includes various mechanisms in communication with one another so as to independently move the energy emitter along the x-, y-, and/or z-axis relative to an underlying tissue (e.g., skin) surface. In an exemplary embodiment, the scanner includes a x-position control mechanism 90 and a y-position control mechanism 92. The x-position control mechanism 90 includes each of a motor, a sensor for sensing the position of the transducer 20 along the x-axis, and a rail 196. The y-position control mechanism 92 includes each of a motor, a sensor for sensing the position of the transducer 20 along the y-axis, and a rail 194. A carriage (not shown) is disposed between the x-axis rail 196 and the y-position control mechanism 92. When the x-position control mechanism 90 moves the carriage along the x-axis, the y-position control mechanism 92 and the transducer assembly 20 move in the x-direction. In this way, the x-position control mechanism 90 and the y-position control mechanism 92 can move the transducer 20 to any location within the window 104. Optionally, the x-axis rail 196 and the y-axis rail 194 are substantially perpendicular to one another. It is possible that one or more of the rails 194, 196 are not orthogonal to one another (e.g., one or both are in the shape of an arc). Those skilled in the art will appreciate that these slidable couplings can be provided in various manners, and all such couplings are within the spirit and scope of the present disclosure.

In this manner, the energy emitter (an ultrasound transducer) 20 can be moved anywhere in an x-y plane (which is typically substantially parallel to the skin surface) to be positioned over a target site. In this way, the x-y movement allows the energy emitter 20 to be positioned anywhere in an x-y plane. Those of ordinary skill in the art will appreciate that various alternative x- and/or y-position control mechanisms are within the spirit and scope of the present disclosure.

As indicated, treatment of various conditions require ultrasound energy to be delivered to a specific depth below a tissue surface so as to target a specific volume of tissue. Also, it is often necessary to deliver a substantially constant amount of energy to numerous such subcutaneous tissue sites located at a substantially uniform depth below the skin surface. One mechanism for repeatedly delivering a substantially constant dose of energy to the various target sites is to position the transmitter 20 at an optimum height above each target site thereby minimizing or substantially eliminating any interference or diminution of energy resulting from the intervening tissue between the tissue surface to the target site.

Referring now to FIG. 8, in an exemplary embodiment, the device 10 can be configured to determine this optimum height for the emitter 20, and can also be configured to position the emitter 20 at this height. Determining the optimum height can be performed in various manners. In an exemplary embodiment, a sensor 96 can be in communication with the tissue site and the control module 82. This sensor 96 can determine some parameter of the tissue indicative of the desired height, and can further communicate this information to the control module 82. Those skilled in the art will appreciate that the device 10 can be configured in various other manners to determine the proper height of the energy emitter 20. The focal depth provided by an emitter 20 together with the desired focal depth of treatment will together determine the height suited to the desired treatment at that location. Optionally, the height may be held at a consistent depth relative to the tissue surface. In another embodiment, for example, where the tissue surface is uneven the focal depth of treatment may be modified at regions of unevenness to ensure that the treatment falls along a substantially level trajectory. In such embodiments the height of the emitter will be moved up and down during treatment of such uneven regions.

As indicated in FIG. 8, in an exemplary embodiment, the scanning mechanism 88 includes a z-position control mechanism 94 configured to position the energy emitter 20 at such a desired height above each tissue site so as to ensure that a substantially constant amount of energy is being delivered to each site. Further, the z-position control mechanism 94 can ensure that energy can be applied to different tissue portions that are located at a substantially uniform depth below the tissue surface (e.g., skin surface). Any such mechanism is within the spirit and scope of the disclosure. For example, in an exemplary embodiment, the z-position control mechanism 94 can exert a compressive pressure to the energy emitter 20 such that the energy emitter 20 is compressed against tissue thereby essentially flattening any intervening tissue between the skin surface and the target site. Applying substantially similar, and preferably identical, compressive pressure to the tissue surfaces above each of the treatment sites, ensures that the emitter 20 is at a known reference height relative to the tissue surface. Accordingly, energy can be deposited at a substantially uniform depth at each site beneath the tissue surface. The uniform compressive pressure can ensure that the amount of intervening tissue between the surface and each of the underlying treatment sites is substantially uniform, thereby allowing the focus to be at a substantially uniform depth. This further allows depositing a substantially uniform dose of energy generated by the transducer 20 to each target site. In the absence of such a pressure, the thickness of the tissue region between the surface and the underlying target site can vary from one target site to another, thereby resulting in variations in the target depths and/or the amount of energy deposited at the tissue targets. However, applying the same compression force above each site can minimize or substantially eliminate any such treatment depth variation.

Referring again to FIG. 9, the z-position mechanism 199 can be configured in various manners to provide such a compression force. For example, the energy emitter 20 can be in communication with a spring or spring-like element located at position 222 configured to compress the energy emitter 20 against the skin surface at some known compression force. In another embodiment, the energy emitter 20 can be in communication with some embodiment of a motor or a pneumatic cylinder located at position 222 configured to repeatedly deliver a pre-determined and/or desired amount of compressive pressure to the energy emitter 20 thereby consistently compressing the energy emitter 20 against each treatment site. In yet another embodiment, a solenoid located at position 222 is configured to repeatedly and predictably compress the energy emitter 20 against the skin at a known compressive pressure so as to provide for substantially consistent delivery of energy to the various treatment sites below the tissue surface. Those skilled in the art will appreciate that various other such mechanisms can be utilized to compress the energy emitter 20 against the tissue surface so as to ensure substantially constant and/or constant delivery of energy to each of the underlying tissue sites. Any such other mechanisms will be located in the region of the z-position control 199. The force exerted by any of the mechanisms at position 222 will be adjusted to enable substantially consistent slidable movement of the energy emitter 20 relative to the tissue surface.

Various other embodiments of z-position control mechanisms 94 can also be employed. For example, after applying a compressive pressure to the tissue surface via the z-position control mechanism, the z-position control mechanism 94 can remove the transducer assembly 20 from target tissue. Thereafter, the transducer assembly 20 can be moved to the next treatment location along the x, y, and/or z-axis.

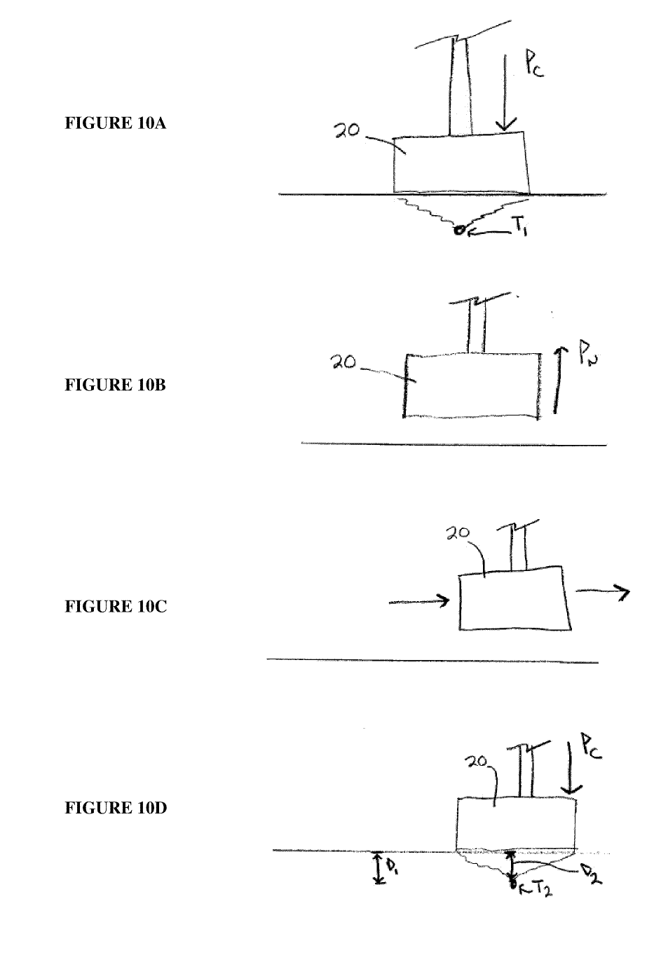

As indicated in FIGS. 10A-10D, in some embodiments, a substantially uniform compressive force P_{C} can be applied to the transducer 20 as the transducer 20 applies ultrasound energy to a first target site T₁ delivered to a depth D₁. Once application of the ultrasound energy to this first target site T₁ is complete, the transducer 20 can be disengaged and lifted (FIG. 10B) from the tissue surface in order to facilitate its movement over the skin surface (FIG. 10C) to another target site T₂. As shown in FIG. 10D, once the transducer is above the second tissue site T₂, substantially the same amount of compressive pressure P_{C} can be reapplied to the transducer 20 so as to accurately target the second tissue site T₂ located at a depth D₂, which is substantially the same depth below the tissue surface relative to T₁ (i.e., D₁≈D₂).

In another embodiment, the device 10 can also include a tightening mechanism configured to, taken alone or in combination with the above-described z-position control mechanism 94, apply a tightening force across all or a portion of the tissue surface of the treatment area thereby effectively minimizing any irregularities and/or inconsistencies found in the region of the various target sites (e.g., inconsistencies found above one or more of the various target sites). As explained above, minimizing or obviating any differences among the various regions of tissue in the region of target tissues (e.g., differences found above the corresponding target tissues) can facilitate delivery of a consistent amount of ultrasound energy to the numerous target sites. In this way, multiple target tissue sites are treated at a substantially even depth.

Referring back to FIG. 9, in an exemplary embodiment, the tightening mechanism can be a frame 100 sized and configured to be positioned substantially flat against a tissue surface. As shown, the frame 100 is rectangular and includes an outer region 102 having a rectangular window 104 disposed therein. The energy from the transducer assembly 20 can be applied to the tissue through the window 104. In use, the frame 100 can be positioned against the tissue surface (e.g., the skin) with such a force so as to provide at least some degree of flattening thereby facilitating the delivery of a consistent amount of energy to each target site. In one embodiment, application of the frame 100 against the tissue results in substantially flattened and/or smoother tissue (e.g., skin). While the outer region 102 and interior window 104 of the frame 100 are shown as rectangular, those of ordinary skill in the art will appreciate that various other shapes and/or configurations (e.g., squares, circles, etc.) are within the spirit and scope of the present disclosure. Also, the interior window 104 can be empty (e.g., void), can be a surface (e.g., glass, polymer, etc.), or, as detailed further below, can include some coupling material or medium (e.g., a gel) specifically configured to provide an optimized coupling of the ultrasound energy from the energy emitter 20 to the target site.

As shown schematically in FIG. 8, following application of the ultrasound dose to one target site, the control module 82 is configured to trigger the scanning mechanism 88 to move the energy emitter 20 to another target site. More specifically, following completion of treatment of the first tissue site, the control module 82 can trigger the z-position control mechanism 94 so as to raise the energy emitter 20 to such a height so as to facilitate lateral movement of the energy emitter along the x-y plane (see FIG. 10C). Subsequently, or substantially concurrent with raising the emitter (e.g., ultrasound transducer) 20 by employing the z-position control mechanism 94, the control module 82 can trigger the scanning mechanism 88 to move the energy emitter 20 via the x- and/or y-position control mechanisms 90, 92 to a position approximately above the next target tissue site. The control module 82 can trigger the z-position control mechanism 94 to position the energy emitter 20 at a desired z-position relative to the tissue surface above the next tissue site, and actuate the emitter 20 to deliver an amount of energy to the target site. As indicated above, typically the z-position control mechanism 94 can be configured such that the energy delivered to the second treatment site is substantially identical to the amount of energy delivered to the first treatment site. This process can then be repeated for each target tissue site of the treatment area.

The presently disclosed device and system also includes various subsystems and components configured to perform and optimize a desired treatment regimen. For example, in an exemplary embodiment, the device 10 can include some form of sensing or imaging technology 80 configured to identify and define a treatment region having a plurality of target tissue sites located therein. Typically, the treatment area is defined as some surface area of tissue and/or some volume of tissue.

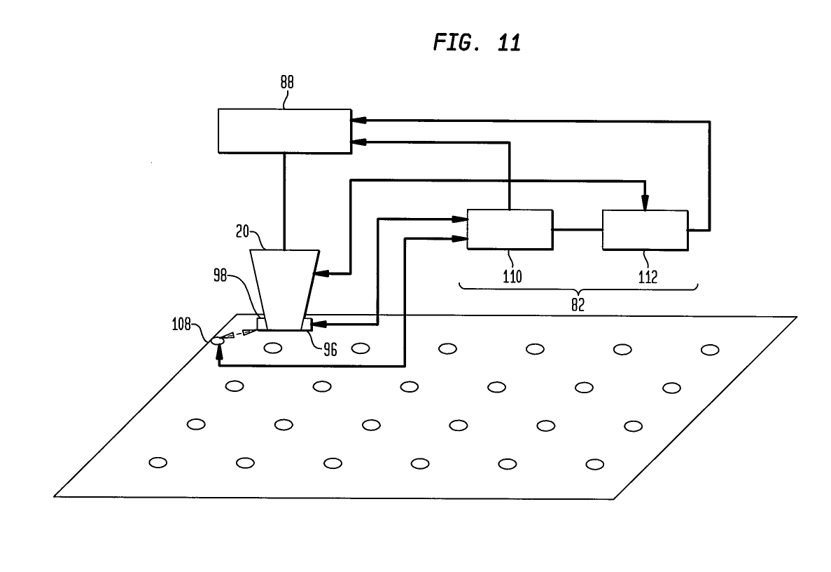

FIG. 11 provides an exemplary embodiment of a feedback mechanism for positioning the transducer 20 at a desired location, determining if the target site needs treatment, and, if treatment is needed, determining the correct dose of energy. As shown, the transducer 20 is coupled to a sensor 96 which is in communication with a reference signal 108. Each of the sensor 96 and the reference signal 108 is in communication with a location determination module 110 of the control module. Based on readings from the sensor 96 and the reference signal 108, the location determination module 112 can determine the exact location of the energy emitter 20 relative the target sites. That is, the reference signal 108 is essentially a stationary point relative to the target sites thereby allowing the location determination module 110 to calculate the x, y, and z location of the transducer 20 based on these readings. In one embodiment, the location determination module 110 can also include a database having the coordinates of each target site. Thus, the location module can be in communication with the scanning mechanism 88 in order to position the scanner at a desired site. Those skilled in the art will appreciate that the reference signal 108 can be any type of sensor configured to provide a signal to the location determination module 110.

Referring still to FIG. 11, the location determination module 110 can also be in communication with a site treatment module 112. That is, once the transducer 20 has been positioned at a target site, the site treatment module 112 can check to see, for example, if this target site has already been treated, if the site is in need of further treatment, the desired energy dose for this particular site, etc. The treatment module can determine if there has been previous treatment in a specific location by any of a number of means including, for example, recording treatment coordinates upon treatment and referencing treatment coordinates with each subsequent treatment, tissue temperature measurement, etc. The treatment module 112, which is also part of the control module 82, can communicate with the scanning mechanism 88 in order to move the transducer 20 to the next target site, apply a suitable compression force to the current site, etc., and the treatment module 112 can also be capable of activating the transducer 20 to fire the desired dose of energy.

Referring now to FIGS. 8 and 9, in an exemplary embodiment, the sensing or imaging mechanism 80 defines the treatment area as corresponding to that area of the tissue surface underlying the transparent or open window 104 of the frame 100. For example, the sensing or imaging mechanism 80 can be in communication with the frame 100 so as to record one or more images of an area of tissue corresponding to the area defined by the window 104 (e.g., a rectangular area as shown in FIG. 9, but optionally other shaped areas determined by the window 104 shape). In some embodiments, a patient may need to be restrained during such treatment area identification. In other embodiments, a sensor can be used which takes a photo of the area defined by the frame and employs an algorithm that alters the scan pattern to compensate for any patient movement with the goal being to capture only the intended scan area despite the patient movement. The photographing sensor can be, for example, an optical mouse. Those of ordinary skill in the art will appreciate that various other mechanisms can be utilized to identify and/or determine a treatment area.

Referring still to FIG. 8, the imaging module 80 can also be in communication with a control module 82 having a memory configured to record various sets of data. The control module 82 can also be in communication with a processor 86 for performing various comparisons and/or calculations. Thus, in those embodiments where the device 10 includes a sensing or imaging mechanism 80 for identifying and/or defining a treatment site (e.g., a target site), this data (e.g., the image, the area of the site, the volume of the site, etc.) can be stored in the memory 84 of control module 82 and is further accessible to the processor 86 for the purpose of performing various calculations that optionally employ one or more algorithm.

In some embodiments, the presently disclosed device and system can identify and/or select a plurality of target tissue sites e.g., 70a, 70b, 70c from the entire treatment area 72 such that selected treatment of these target sites 70a, 70b, 70c can provide the desired therapeutic effect to the entire treatment area 72. For example, in one embodiment, at least one algorithm can be stored within the control module 82. The stored algorithm can generate some pre-determined pattern and/or number of target tissue sites in view of the previously recorded treatment area. For example, as shown in FIGS. 7A and 7B, the control module 82 can determine a grid-like pattern of tissue target sites 70 dispersed substantially evenly throughout the treatment area 72.

In an alternative embodiment, the control module 82 can determine the target sites 70 based on some analysis of information relating to the treatment area. For example, the control module 82 may identify various areas to be treated based on some measured parameter, such as, for example, discoloration, progression of skin condition (e.g., acne), etc. As will be apparent to those of ordinary skill in the art, any such procedures for selecting target tissue areas is within the spirit and scope of the present disclosure.

In an exemplary embodiment, the control module 82 of the device 10 is in communication with the scanning mechanism 88 thereby allowing the control module 82 to position the energy emitter at a desired x-, y-, and/or z-position for a desired period of time so as to effect treatment. That is, upon identifying the number and location of target tissue sites 70 within the treatment area 72, the control module 82 can be configured to activate the scanning mechanism 88 so as to position the energy emitter 20 at a location substantially above the target tissue site 70. Once the energy emitter 20 is positioned at a location substantially above the target tissue site 70 (or concurrently with the x- and y-positioning of the energy emitter 20), the control module 82 can further be configured to active the z-position control mechanism 94 thereby allowing for the energy emitter 20 to deliver a known amount of ultrasound energy to a tissue site located at some depth D below the tissue surface. At this stage, the control module 82 can now be configured to initiate the treatment protocol thereby firing the energy emitter 20 so as to deliver the desired dose (e.g., desired frequency, pulse width, etc.) of ultrasound energy to the target site 70 (e.g., 70a, 70b, 70c of FIG. 8).

Referring still to FIG. 8, in some embodiments, the device 20 can also include one or more sensors 96, 98 configured to determine some parameter(s) associated with the target tissue and/or the tissue surface which is indicative of the need for further treatment. That is, following application of ultrasound energy to the target site 70a, a sensor 98 can determine some treatment parameter (e.g., temperature of tissue surface, etc.), and communicate this parameter to the control module 82 which can then determine the treatment status for that site. That is, the control module 82 can determine, for example, whether that site 70a needs to be targeted again, and if so, whether that site should be immediately re-targeted, or if the energy emitter 20 should return to that site after targeting other treatment sites e.g., 70b. Additionally, in those cases where treatment is to be repeated, the control module 82 can determine (and implement) whether the same dose of ultrasound energy should be applied during this repeated treatment, or whether a new ultrasound dose should be applied to the site to achieve the desired therapeutic effect.

The presently disclosed device can include various types and/or configurations of such sensors 98. For example, the sensors 98 can be temperature sensors configured to determine the temperature at the tissue surface (e.g., skin surface). The sensor can detect visible damage by optical means (e.g., pigment change, inflammation detection). Those of ordinary skill in the art will appreciate that various other sensors are within the spirit and scope of the present disclosure.

In some embodiments, referring now to FIGS. 8 and 11, the control module 82 can implement a registration and tracking protocol that can be utilized to ensure that each target site 70 is exposed to the ultrasound energy a pre-defined number of times (e.g., only once). By way of example, with reference to FIG. 11, at the beginning of a scan, a location of the transducer's 20 tip can be determined relative to a reference location of the frame, as the transducer 20 is moved to a target region, a motion sensor 98 coupled to the transducer's housing can determine x and y distance traveled by the transducer relative to the reference location. The x and y coordinates of the target site 70a relative to the reference location can then be recorded, e.g., in the memory 84 of the control module 82. Upon completion of the application of the ultrasound energy to that target site, a log maintained by the control module 82 can be updated to indicate that a treatment dose has been applied to that site. In one embodiment, a table can be consulted to determine whether an individual site has been treated previously, if not, the control module 82 can activate the transducer 20 to treat that particular site.

While the above embodiments discuss delivery energy to discrete location thereby providing discrete treatment sites 70a, the device and methods can also be configured to provide continuous treatment. Thus, in some embodiments the transducer 20 can deliver energy while moving relative to the tissue surface thereby producing a treatment site of any desired configuration (e.g., a line-like lesion).

One challenge associated with ultrasound use is achieving good coupling (e.g., contact) of ultrasound energy into the target tissue. In order to couple ultrasonic energy from a transducer to the tissue, a coupling medium can be used. In some embodiments, a coupling medium (e.g., a gel) can be continuously disposed between the ultrasound transducer device and the tissue surface as the device is scanned over the tissue. In one embodiment, the coupling medium is selected such that the speed of sound through the medium is substantially the same as the speed of sound through the tissue. In order for a coupling medium to be effective, the medium should be preferably substantially free from imperfections such as, for example, air gaps, bubbles, or voids. In the case of a focusing transducer, effective coupling is challenging because the transducer is usually a concave shape, and is positioned at some distance away from the surface of the tissue being treated.

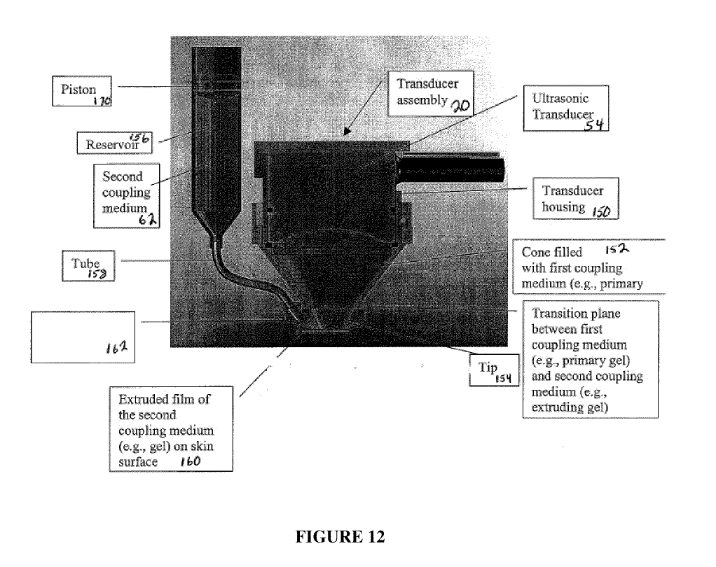

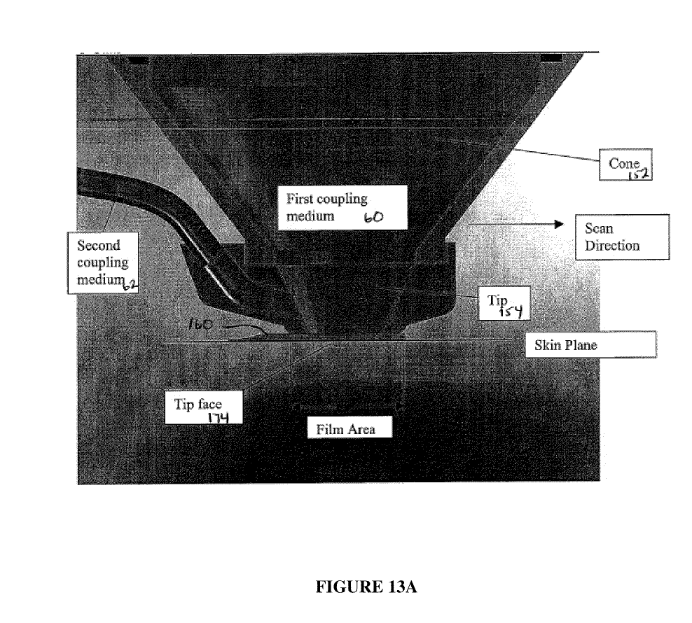

In one embodiment, the presently disclosed device provides consistent and effective coupling of ultrasonic energy from a transducer to tissue via a substantially constant coupling of the ultrasonic energy from the transducer to the tissue. FIG. 12 provides a cross-sectional view of an ultrasonic transducer 54, having a housing 150, with an attached cone 152 and tip 154. The cone 152 is filled with a first coupling medium 60 (e.g., a gel or any other substance with acoustic impedance close to the acoustic impedance of water including commercially available gels such as Aquasonic Ultrasound Gel, see FIG. 6). The first coupling medium 60 housed inside the cone 152 contacts the ultrasonic transducer 54 surface and extends downward to a plane near the tip 154. A portion of the tip 154 extends the cavity that exists between the transducer 54 surface and the surface of the tissue being treated, which focuses ultrasound at the desired depth relative to the tissue surface and the coupling medium couples the ultrasound energy from the transducer to the tissue surface.

The extension 162 can be a conduit that connects the reservoir 156 (e.g., via tubing 158) to the tip 154. At least a portion of the reservoir 156 is filled with another coupling medium (i.e., a second coupling medium) 62. The second coupling medium 62 (e.g., a gel) can have a viscosity which is low enough such that it can be driven (e.g., via gravity and/or mechanically driven) from the reservoir 156 to the tip 154. Optionally, the viscosity of the second coupling medium 62 is high enough such that is does not flow without being driven out of the reservoir. In one embodiment, the first coupling medium 60 (e.g., the primary gel) is the same viscosity as the second coupling medium 62. In another embodiment, the first coupling medium 60 has a higher viscosity than the second coupling medium 62. Where the first coupling medium 60 has a relatively higher viscosity than the second coupling medium 62, the viscosity of the first coupling medium 60 will be up to about 500,000 cPs. In one embodiment, the first coupling medium 60 has a relatively thick gel-like agar or gelatin-like consistency. The first coupling medium 60 may be molded and shaped such that the second coupling medium 62 is in contact with the transducer 54. The second coupling medium 62 can be capable of forming an extruded film 160 which has a viscosity that ranges from about 80,000 cPs to about 100,000 cPs. In one embodiment, both the first coupling medium 60 and the second coupling medium 62 have a viscosity that ranges from about 80,000 cPs to about 100,000 cPs at [temp/pressure] conditions.

In use, referring again to FIG. 12, the second coupling medium 62 can be driven through the transducer assembly 20 via gravity through a tube 158 and into an extension 162 and through the tip 154. Alternatively, the second coupling medium 62 can be driven through the transducer assembly 20 via a drive source, such as a piston 170. In one embodiment, one or more of the cone 152, tip 154, tube 158, first coupling medium 60, and the second coupling medium 62 are disposable, and the tube 158 and/or the extension tip 154 are provided charged with the second coupling medium 62. Pre-charging the tube 158 and the extension tip 154 with a second coupling medium62 can avoid undesirable air entrapment.

Referring to FIG. 6, the transducer 54 can include a listening transducer 56 that provides a testing pulse to reflect a signal to determine the presence or absence of bone. Ultrasound can damage bone and is thereby a safety concern. The listening transducer 56 can be employed to determine the presence or absence of bone to ensure that the device does not fire on a bone interface. Alternatively or in addition, z-position control, discussed above, can be employed together with the listening transducer (e.g., a sonic transducer) 56 to determine the suitable depth of treatment in the z direction thereby to avoid bone damage. The listening transducer 56 can be employed for treatment and/or for feedback control during treatment. The transducer 54 focuses energy in the z-direction at a focus point.

In an exemplary embodiment, the transducer assembly 20 provides a substantially constantly refreshed supply of a second coupling medium (e.g., extruding gel) 62, substantially avoids breakdown, cavitation bubbles, and/or particulates which otherwise might be present near the concentrated energy focus of the ultrasonic beam. In addition, the supply of an amount of fresh second coupling medium 62 can ensure that the tip 154 and the tissue surface do not touch.

The second coupling medium 62 can serve various functions. For example, the second coupling medium 62 can enhance the coupling of the ultrasound energy from the transducer 54 to the target tissue (e.g., skin). The second coupling medium 62 can also provide some measure of cooling, by displacing gel that might be heated by proximity to the skin being treated. The second coupling medium 62 can also provide a low friction film between the tip 154 and skin, which can be particularly beneficial in scanning operations.

In the application of an ultrasonic scanner system that does not utilize an extruded gel as disclosed herein, where a transducer is moved in discrete steps on the tissue (e.g., skin) and then pulsed, the spatial uniformity of the resulting pulse pattern (array of treatment dots) depends in part on the skin remaining stable. A precise translation of the pulse might not be completely effective if the relatively soft tissue distorts laterally while the head is being scanned. If maintained, such contact between the tip and the tissue surface could provide friction and thereby disrupt the treatment. An alternative to scanning would be to lift the head, reposition the transducer, and lower it between pulses, but that requires a large amount of down time in contrast to scanning, and is potentially unpleasant to the patient (e.g., because of the time required to conduct the treatment procedure). Such up and down “pick in place” type movements could also risk entrapped air bubbles near the tip.

Referring again to FIGS. 6 and 12, the constant metered extrusion of a second coupling medium 62 causes a thin film 160 to form between the tissue surface (e.g., the skin) and the face of the tip 154. The shape of the tip's face and/or the properties of the second coupling medium 62 can be selected to utilize and/or to optimize the hydrostatic bearing between the tip 154 and the film 160. The substantially constant fresh supply of the second coupling medium 62 is provided to a critical area that ensures that the tip 154 and the target tissue surface (e.g., the skin) do not touch. To avoid contact between the transducer assembly 20 and the target tissue there should preferably be enough pressure of the transducer 20 and film 160 of the second coupling medium 62 to ensure a hydrostatic bearing. In a hydrostatic bearing, fluid is pumped into the gap between two moving components. The pressure with which such a fluid is pumped, and the area of the contact determine how thick the film shall be and how much external force it can withstand while maintaining a film 160. If the two surfaces are moved laterally with respect to each other, the frictional component will be related to the shearing properties of the fluid, which can be extremely low, especially compared to the coefficient of friction between two solids, which is usually in the range of about 0.05 to about 0.4

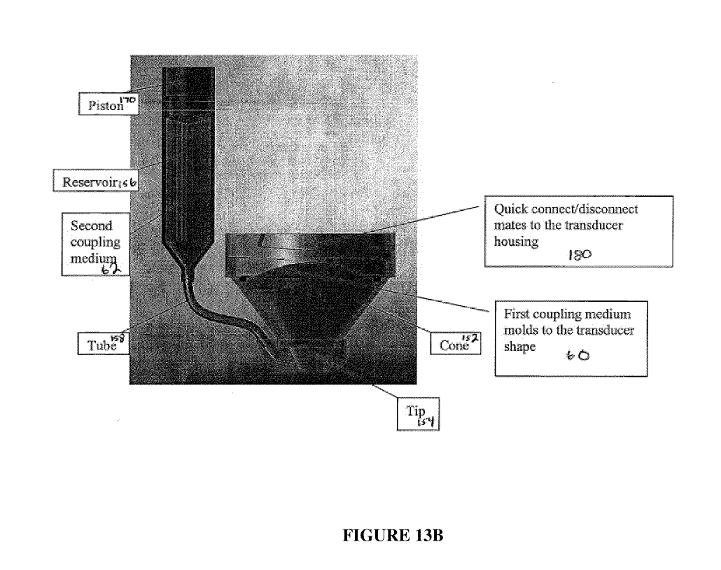

In one embodiment, referring to FIG. 13A, the face of the transducer assembly tip 154 is scanned in the scan direction across the tissue surface (e.g., skin) plane with an applied downward force sufficient to maintain contact between the tip face 174 and the film 160. As the gel 62 extrudes and a film 160 is created, any lateral friction forces will be minimized and/or eliminated. Minimizing and/or eliminating the lateral forces will thereby minimize and/or avoid lateral skin distortion, and distortion of the scan pattern will be substantially and/or completely avoided. In this manner, a scan of successive pulses might be done faster, and with less impact to the patient.

An additional benefit of the extruding gel 62 is to provide a visual reference of areas already scanned. In these embodiments, pulse marks are not necessarily visually evident on the skin at the areas previously scanned. This visual reference can be enhanced via color or reflective additives in the gel. In this way, any unintended, repeated scanning can be avoided and/or the area of prior treatment can be made evident to the practitioner.

In almost all cases, any surface of a medical device that contacts a subject must be cleaned, sterilized, or replaced between individual subjects. In one embodiment, the tip 154 of this device can be cleaned with sterilizing solutions, then sufficient amounts of gel can be pumped through the tip to ensure that only fresh material is exposed to the next patient. Alternatively, all or at least a portion of the device may be disposable after a single treatment. For example, the tip 154 might be disposable and replaced between patients. Alternatively, the cone 152 and the tip 154 can be replaced after each patient (or at any other desired time).

FIG. 13B shows an embodiment where the disposable components include the cone 152, tip 154, a first coupling medium 60, a second coupling medium 62, a tube 158, and a reservoir 156 wherein any or all of these components can be disposable and/or replaceable at any stage of treatment and/or between patients. FIG. 13B also shows a disconnect coupling 180 for the cone 152 which mates to the transducer housing 150. The transducer housing 150 can be fixed in place to enable quick connection to and disconnection from the cone 152. Those skilled in the art will appreciate that various couplings and disconnection couplings (e.g., snap fits) are within the spirit and scope of the present disclosure.

In many embodiments, efficient ultrasound delivery requires the first coupling medium 60 to be in direct contact with the transducer 54. Referring to FIG. 12, it is desirable to minimize and/or substantially eliminate all air gaps and/or bubbles at the border or interface between the transducer 54 and the first coupling medium 60. In one embodiment, the first coupling medium 60 is a relatively high viscosity gel (e.g., from about 80,000 cPs to about 100,000 cPs, or up to about 500,000 cPs), molded to a shape complementary to the transducer. In this way, the cone 152 can be simply attached to the cone tip 154, and coupling between the first coupling medium 60 in the cone 152 and the transducer 54 can be achieved without the necessity of refilling fluids and purging bubbles at the border between the transducer 54 and the first coupling medium 60. In one embodiment, any or all of the reservoir 156, piston 170, tube 158, and tip 154 are also disposable. The reservoir 156 is charged with the second coupling medium 62, a gel that is to be extruded having a viscosity lower than the viscosity of the first coupling medium 60. A removable cap or foil lid (not shown) will prevent leakage from the tip 154 and/or drying of the second coupling medium 62 before use.

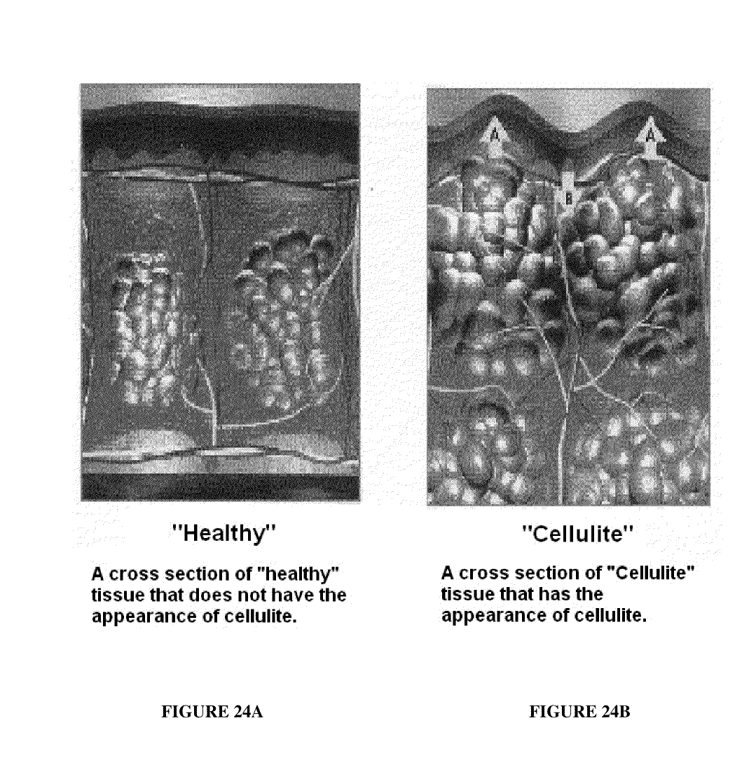

Referring now to FIGS. 24A and 24B, in one embodiment cellulite may be treated via ultrasound. Referring to FIG. 24B, cellulite is characterized by strands of connective tissue (B) that pull skin inwards thus creating a “dimpled” or “tufted” (e.g., mattress-like”) appearance of the skin surface caused, for example, by tissue that is not pulled inwards that instead bows out (A). Referring now to FIG. 24A, “healthy” tissue likewise has strands of connective tissue, but in healthy tissue that does not have the cellulite appearance the connective tissue is not pulled in or tight. The cellulite appearance can occur in relatively thin individuals who have relatively tight strands of connective tissue in a given area.

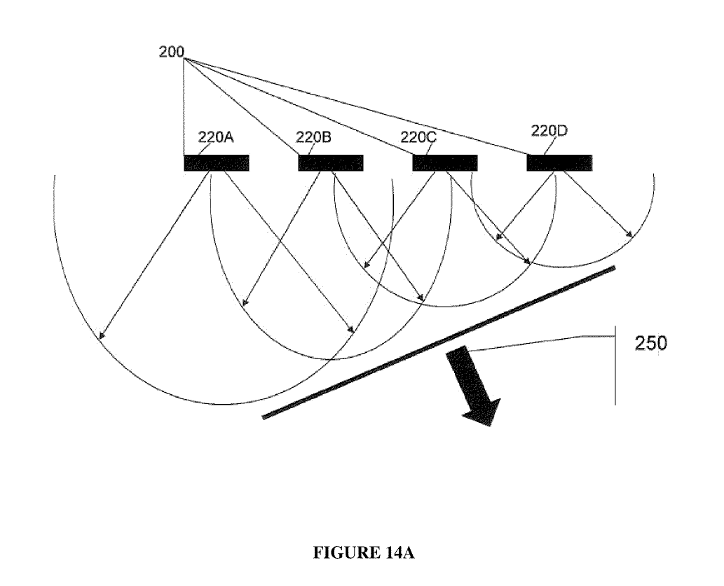

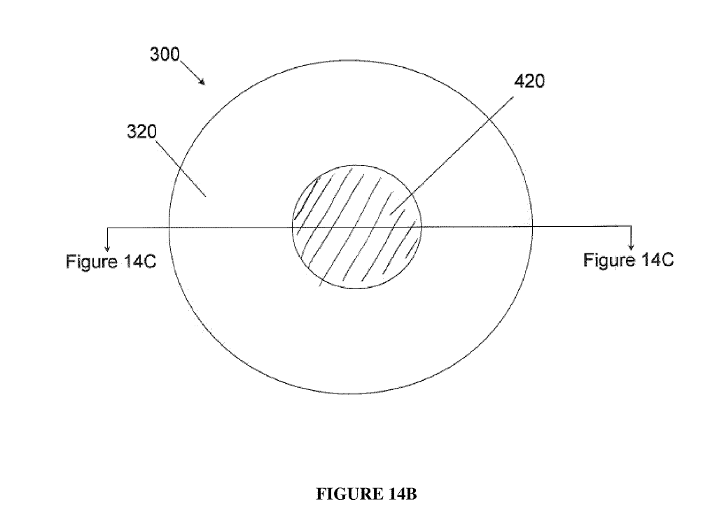

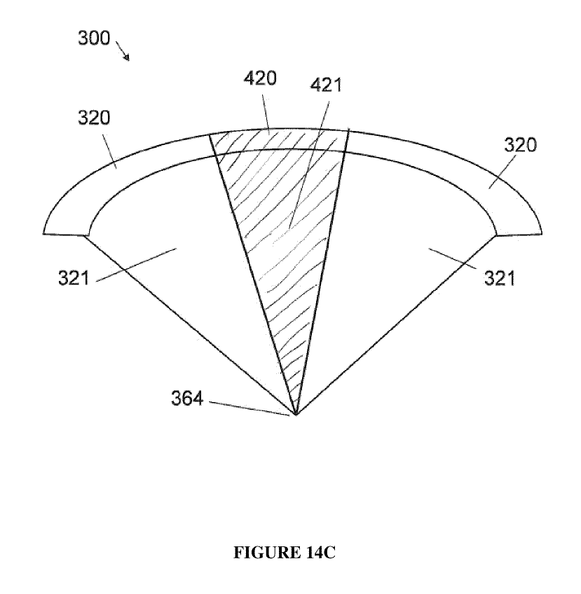

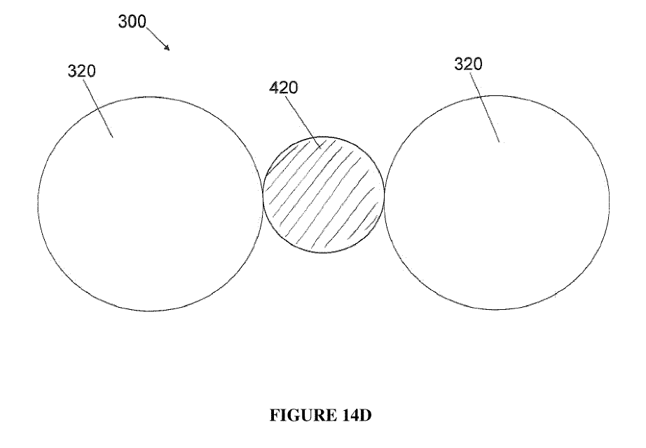

By applying ultrasound energy to these strands of connective tissue, the tension and/or pressure of the strands of connective tissue can be relaxed and/or eliminated such that the pull of the strands of connective tissue inwards (B) is lessened and/or eliminated. Strands can be thermally effected to be relaxed. For example, the ultrasound energy can be employed to thermally denature one or more structural components such as a protein of the strand. The thermal effect of the ultrasound energy can be used to sever one or more strand such that no tension remains left in the strand. In this way, the “dimpled” or “tufted” appearance on the surface of the skin is diminished or removed (e.g., smoothed out). Thus, ultrasound can be employed to improve the appearance of cellulite. The locations of the strands can be detected with diagnostic ultrasound pulse or another diagnostic technique. In some embodiments, an array of transducers (e.g., a phased array of transducers) can be used (e.g., instead of a scanner), to provide manipulation of the focal point of ultrasonic energy delivery. An array of transducers may be used in addition to a means for mechanical movement of the transducer. Optionally, an array of transducers may be employed without mechanical movement of the transducer. FIG. 14A shows a phased array 200 of transducers. The phased array 200 is made up of a plurality of transducer elements 220A, 220B, 220C, and 220D, e.g., here four transducer elements make up the phased array 200 of transducers. The phased array 200 of transducer elements create a synthetic wave front. The phased array 200 of transducer elements can be employed to apply ultrasound energy to a subject's tissue.

### Producing Coagulation at or Below the Subject's Pain Tolerance Threshold

In order to get a therapeutic effect in tissue one wants to produce lesions of coagulation with a coagulation radius that ranges from about 0.1 mm to about 0.8 mm. Previously such a range of radii (e.g., from about 0.1 mm to about 0.8 mm) was only achievable with a level of pain experienced by the subject that would require at least topical (and more commonly, local) anesthesia. It is desirable to coagulate tissue with a coagulation radius that ranges from about 0.1 mm to about 0.8 mm via ultrasound energy in a manner that induces pain below a threshold tolerable by subjects such that anesthesia may be avoided.

The lesions of coagulation (e.g., the coagulation zone(s)) produced by the method disclosed herein have a shape that is close to an ellipsoid of axial symmetry, with low asphericity (e.g., is substantially spherical). The coagulation radius is the radius of a hypothetical ideal sphere that has the same volume as an actual lesion of coagulation (e.g., the coagulation zone(s)) but that has been approximated as a hypothetical ideal sphere.

In accordance with this method, a combination of treatment parameters that delivers a treatment volume with a coagulation radius that ranges from about 0.1 mm to about 0.8 mm while inducing pain that is below a tolerability threshold such that anesthesia is unnecessary (or may be avoided).

Pain can be measured on a subjective scale of 1 to 10 where 1 is defined as absence of pain and 10 is defined is the worst pain imaginable by the respondent (e.g., the subject). In one embodiment, a pulse width of 15 ms or less, e.g., from about 5 ms to about 15 ms was employed and achieved a pain level of between 3 and 1 (total of 6 subjects), which is viewed as generally “painless” or “negligible pain.” In another embodiment, a pulse width of from about 15 ms to about 50 ms was employed and achieved a pain level of between 5 and 1 (total of 6 subjects), which is viewed at generally as a “tolerable” level of pain that does not require anesthesia.

In one embodiment, dermis tissue was treated with a power level of 250 Watts, a frequency of 5 MHz, and at a pulse width of 15 ms or less (e.g., from about 5 ms to about 15 ms) to achieve an average coagulation treatment radius of between about 0.5 mm to about 0.8 mm (total of 3 subjects) and the subjects rated the pain level between 1 and 3, which is viewed as “painless” or “negligible pain”.

Ranges of parameters suitable for use with the following pulse width's and/or power levels are, for example:

(A) a pulse width range of from about 0.1 ms to about 5 ms; a power level range of from about 10 Watts to about 150 Watts; a frequency range of from about 0.7 MHz to about 20 MHz or a frequency range of from about 2 MHz to about 8 MHz.

(B) a pulse width range of from about 5 ms to about 50 ms; a power level range of from about 150 Watts to about 500 Watts; a frequency range of from about 0.7 MHz to about 20 MHz or a frequency range of from about 2 MHz to about 8 MHz.

(C) a pulse width range of from about 10 ms to about 30 ms; a power level range of from about 200 Watts to about 300 Watts; a frequency range of from about 0.7 MHz to about 20 MHz or frequency range of from about 2 MHz to about 8 MHz.

### Heating and Cavitation in Combination

In one embodiment a transducer is employed to heat a region, e.g., a volume, of tissue to a temperature within a desired temperature range. The heated region can be raised from the temperature range of normal physiological body temperature (e.g., from about 30° C. to about 38° C.) to a temperature in the range of from about 30° C. to about 65° C., from about 40° C. to about 65° C., or from about 40° C. to about 55° C. Thus, the acoustic pulses can heat the region of tissue to cause a temperature rise in the region, e.g., a volume, of tissue of at least 5° C. or from about 5° C. to about 35° C. Thus, transducer heats a volume of tissue to a temperature within the range of from about 30° C. to about 65° C. When heating the volume of tissue the transducer applies a frequency that ranges from about 0.7 MHz to about 20 MHz. The heating acoustic pulse(s) applied by the transducer has a power density of from about 500 W/cm² to about 5,000 W/cm² and has an energy density of from about 2.5 J/cm² to about 25 J/cm².

A transducer is employed to produce cavitation activity in previously heated tissue region (e.g., in the volume of tissue), more specifically, in the tissue region having a temperature raised by at least about 5° C. by the heating acoustic pulse(s) applied by the transducer transducer employed to heat the region of tissue. To produce cavitation activity, the transducer applies a cavitation acoustic pulse with a frequency that ranges from about 20 kHz to about 700 kHz. The cavitation acoustic pulse(s) applied by the transducer has a power density of from about 40 W/cm² to about 800 W/cm² and has an energy density of from about 4 J/cm² to about 80 J/cm².

The clinical endpoint of cavitation will vary based on the treatment application. For example, with tattoo removal the clinical endpoint may be a visible blanching of the appearance of the tattoo and/or a change in color of the tattoo in the skin. Combining heating and cavitation as disclosed herein can be used for any of a number of clinical applications such as, for example, 1) tattoo removal 2) fat reduction 3) cellulite treatment 4) skin tightening and 5) treatment of malformations such as lesions, tumors etc.

In one embodiment, a single transducer is employed to apply at least one heating acoustic pulse to first heat the volume of tissue and then the transducer is employed to apply at least one cavitation acoustic pulse to produce cavitation activity in the heated volume of tissue. More specifically, the transducer first applies at least one heating acoustic pulse having a frequency that ranges from about 0.7 MHz to about 20 MHz to heat the volume tissue by at least about 5° C. or from about 5° C. to about 35° C. Once the tissue is heated the temperature of the tissue region (e.g., the volume of tissue) is within the range from about 30° C. to about 65° C., within the range of from about 40° C. to about 65° C., and/or more within the range of from about 40° C. to about 55° C. The same transducer is then switched to a lower frequency such that it applies a frequency that ranges from about 20 kHz to about 700 kHz to produce cavitation activity in the heated volume of tissue (in the region of tissue). The temperature of the volume of tissue may be monitored by any of a number of suitable methods including, for example, one or more of: contact temperature measurements at the skin surface, non-contact (e.g., IR) measurements at the skin surface, invasive (needle) measurements of temperature inside the tissue, and/or non-invasive (e.g., phase-sensitive US, opto-acoustic) measurements of temperature inside the tissue.

Cavitation activity can be determined by various means including acoustical means and optical means. In one embodiment, cavitation activity is determined by acoustical means that employ a hydro phone which transforms an acoustic signal to an electrical signal. The electrical signal produced via the hydro phone informs the user that cavitation activity has been achieved. The amount of cavitation activity can be detected by the electrical signal that is produced. In another embodiment, the cavitation activity is determined by optical means such as by employing sonoluminescence which detects light emitted when cavitation bubbles collapse. The quantity of detected light emission can enable determination of the amount of cavitation activity produced by the transducer. The clinical endpoint of cavitation activity may vary by treatment application (e.g., tattoo treatment can have a cavitation activity endpoint that differs from the cavitation activity endpoint of cellulite treatment). The cavitation activity endpoint may be determined by visually determining when the clinical endpoint has been achieved (e.g., when the tattoo has been blanched). In some embodiments, the cavitation activity endpoint is determined by the quantity of cavitation activity, which may be determined by electrical signal and/or by optical means, for example.

In another embodiment, multiple transducers (i.e., two or more transducers) are employed such that one transducer applies a frequency that ranges from about 0.7 MHz to about 20 MHz to heat the volume tissue and another transducer (i.e., a separate transducer) applies a frequency that ranges from about 20 kHz to about 700 kHz to produce cavitation activity in the heated volume of tissue. In one embodiment, a first transducer is relatively larger than the second smaller transducer.

Optionally, a composite transducer is made up of multiple transducers. For example, in one embodiment, one or more relatively high-frequency transducers and one or more relatively low-frequency transducers are incorporated into a composite transducer. For example, referring now to FIGS. 14B and 14C, one composite transducer 300 includes a smaller transducer 420 incorporated into a central opening of the larger transducer 320. For example, the larger transducer 320 can have an aperture configured to hold the smaller transducer 420. For example, the relatively smaller transducer 420 can have a relatively higher frequency range (e.g., in the MHz range of frequencies) and can be employed to elevate the temperature of a subject's tissue. The relatively larger transducer 320 can have a relatively lower frequency range (e.g., in the kHz range of frequencies) and can be employed to produce cavitation in a subject's tissue. Referring now to FIG. 14C the relatively smaller transducer 420 and the relatively larger transducer 320 each propagate ultrasound acoustic signals (waves) 321 and 421 that can be applied to a subject's tissue. For example, the acoustic waves 321 and 421 propagated by the co-located transducers 320 and 420 can be co-focused at a point 364 within the subject's tissue being treated.

In another embodiment, referring to FIG. 14D, another composite transducer 300 is made up of co-located transducers where a relatively smaller transducer 320 is flanked by two relatively larger transducers 420 each on opposite sides of the smaller transducer 320 along a horizontal axis.

Heating and cavitation may be done sequentially, simultaneously, and/or sequentially with some overlap such that heating occurs and during the heating process cavitation of the treatment area beings. Sequential, simultaneous, and/or sequential with some overlap can be conducted with a single transducer or with multiple transducers.

Without being bound to any single theory it is believed that elevating the temperature of the tissue modifies the cavitation activity. It is known that the intensity of cavitation in aqueous media varies with temperature of the aqueous media such that cavitation activity generally increases as temperature increases up to a threshold amount. Thus, combining heating of tissue via a frequency applied with a transducer with inducing cavitation activity in tissue via frequency applied with the transducer that heated the tissue or with a different transducer is expected to intensify the cavitation activity of the tissue at the same acoustic power level, therefore producing fewer side effects. Combining heating with acoustic energy and inducing cavitation with acoustic energy enables the threshold for cavitation to be reduced so that cavitation can be induced with a lower power density and a lower energy density than would be required in the absence of heating. In this way, by employing heating acoustic pulse(s) with cavitation acoustic pulse(s) fewer and/or less intense side effects of cavitation result.

In one embodiment of a method for treating tissue a heating acoustic pulse is applied to a region of tissue (e.g., a volume of tissue) to raise the temperature of the region of tissue by at least 5° C. One or more heating acoustic pulse can have a frequency of from about 0.7 MHz to about 20 MHz and have a power density and an energy density sufficient to raise the temperature in the region of tissue at least 5° C. (e.g., from about 5° C. to about 35° C.). One or more cavitation acoustic pulses are applied to the region of tissue (e.g., a volume of the tissue) with a frequency range of from about 20 kHz to about 700 kHz with a power density and an energy density sufficient to induce cavitation in the region of tissue. Optionally, the heating acoustic pulse has a power density of from about 500 W/cm² to about 5,000 W/cm² and has an energy density of from about 2.5 J/cm² to about 25 J/cm² and the cavitation acoustic pulse has a power density of from about 40 W/cm² to about 800 W/cm² and has an energy density of from about 4 J/cm² to about 80 J/cm².

Cavitation activity is determined through the concentration of cavitation bubbles in a cavitation zone. It is difficult to initiate cavitation in certain regions of tissue, for example, initiating cavitation in a dense and viscous area of the skin tissue can be challenging. In tissue that has blood vessels and capillaries the blood vessels and/or capillaries would be the first places affected by the cavitation activity (which may not be a desired outcome). In difficult treatment areas, for example, areas where there are blood vessels and/or capillaries that are not the targeted areas for treatment, the transducer energy can be focused at the target areas (i.e., focused at tissue such that the transducer energy avoids capillaries and blood vessels). Such focusing can be achieved by employing a focusing transducer and/or an array of transducers with synthetic aperture capabilities. A focusing acoustic transducer is described herein in relation to FIGS. 2, 3, and 6, for example.

### Experiments

To illustrate the efficacy of the above methods for treating tissue by application of ultrasound energy and for preliminary safety evaluation of such tissue treatments the results of a number of in-vitro experiments are presented below. Direct and indirect evidence of the formation of coagulation zones (lesions) was obtained. Coagulation lesions were created ex vivo in dermis and fatty tissue of pig and human skin. The appearance of the lesions in vitro was demonstrated with H&E and NBTC staining techniques and photography using a microscope. The following parameters were employed for the ultrasound pulses:

Frequency: 3.4 and 5 MHz,

Electrical power max: 250 W,

Acoustic spatial focal peak intensity: 800-1000 W/cm²,

Pulse widths: 10-70 ms.

Experiment 1: Treatment of Pig Skin

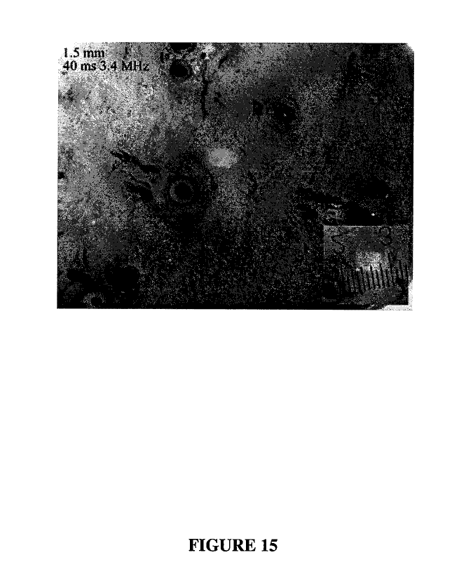

Samples of pig skin were sonicated with 3.4 MHz focused ultrasound with a single pulse of 40 ms in duration. The ultrasound energy corresponded to the spatial peak intensity of about 800 to about 1000 W/cm² (without considering attenuation and nonlinear effects in tissue and water). The F-number was 0.9 and the focal spot was located at another 2.5 mm below the skin surface. As shown in FIG. 15, at these chosen ultrasound settings, coagulation lesions were created below the epidermis.

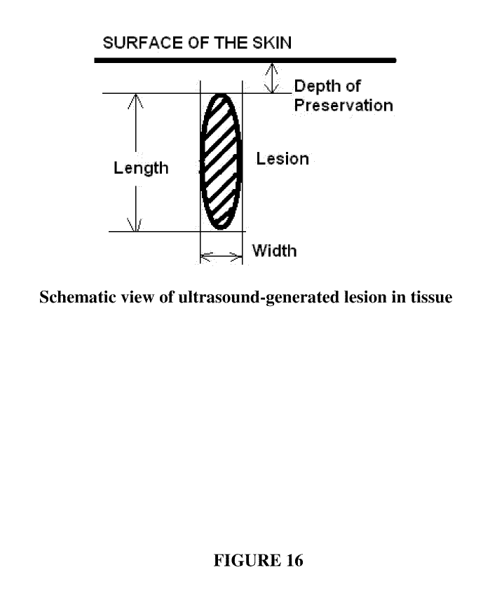

The first signs of coagulation appeared at about 1 mm to about 2 mm under the skin surface and disappeared beneath about 4 mm to about 5 mm of the skin surface, the maximum diameter of the coagulation spot ranged from about 0.8 mm to about 1.5 mm, or about 1 mm. The depth (e.g., the z direction) of the coagulation lesion ranged from about 4 mm to about 1 mm. The length of the lesion (FIG. 16) was from about 1.5 mm to about 3 mm. FIG. 16 presents a schematic view of ultrasound-generated lesion in tissue and Table 1 contains results from several samples of skin to which ultrasound energy was applied with the same ultrasound parameters to verify reproducibility of the method. On reason for the variability in results shown in Table 1 is the difference between skin specimens.

TABLE 1
Coagulation signs detected in pig tissue after 40 ms of sonication

| | First coagulation | | Last sign of |
|---|---|---|---|
| | detected, mm | Maximum diameter | coagulation, mm |
| Sample | (below the surface | of the coagulation | (below the surface |
| # | of the skin) | spot, mm | of the skin) |
| 3 | 1.0-1.5 | 0.8 | 3.5-4.0 |
| 4 | 1.0-1.5 | 1.1 | 2.0-2.5 |
| 5 | 2.0-2.5 | 1.5 | 4.0-4.5 |
| 6 | 1.5-2.0 | 1.0 | 3.5-4.0 |

### Experiment 2. Safe Ultrasound Exposure Times for Different Focal Places

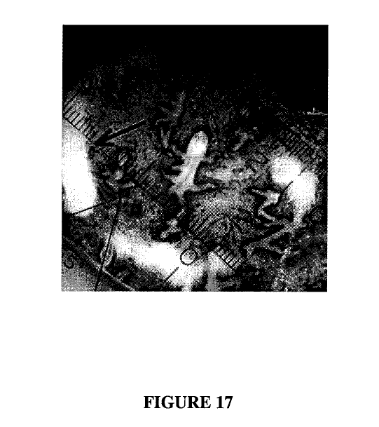

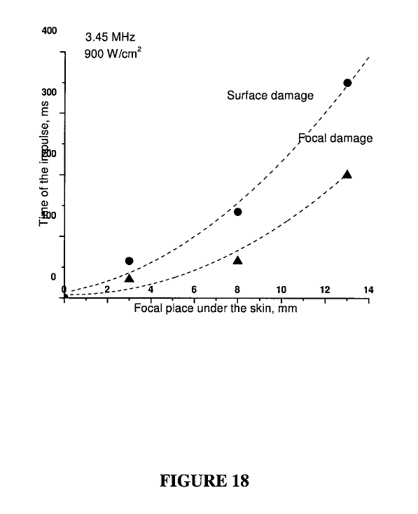

When sonication times go above certain values, it can result in skin burns (FIG. 17). In order to generate a lesion in tissue without causing skin burns proper exposure times need to be selected. We determined that for the lesions located deep in the tissue, the length of ultrasound impulses which do not cause skin burns can be as much as hundreds of milliseconds (see FIG. 18).

Therefore, at selected acoustical power and transducer configurations, two important parameters are the time of the surface damage and focal damage. Threshold time of the surface damage is important for safety of the technique and the focal damage is important for efficacy of the treatment. Both of the parameters depend on the focal place under the skin (e.g., the focal depth).

### Experiment 3. Treatment of Human Skin

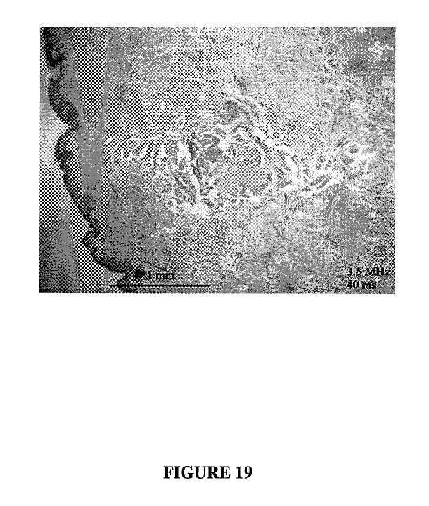

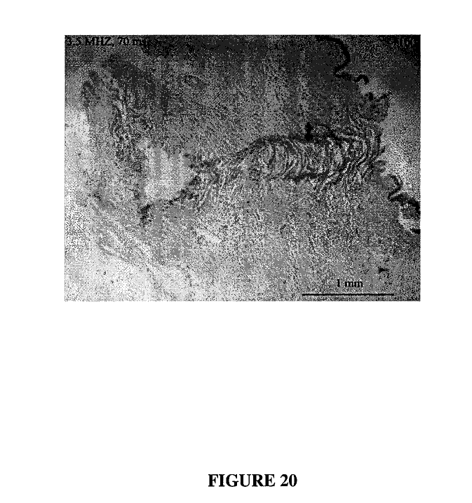

A sample of a freshly excised 1 cm thick piece of human abdomen skin was sonicated with 3.4 MHz focused ultrasound. Temperature below the skin sample was kept at 37° C. (the temperature of the human body), the temperature at the surface of the skin was 27° C. The location of the sonicated spot was removed with a biopsy punch, and stained with the H&E technique. Device parameters were about 250 W of electrical output power, 900 W/cm² of acoustic spatial focal peak intensity and 40 ms pulse lengths. The focal depth was 2 mm below the skin surface and the F-number was 0.9. As shown in FIG. 19, at these chosen parameters, focused ultrasound produced coagulation with no detected damage to the epidermis. Without changing location of the focal area such that the focal area was located ˜2 mm beneath the surface of skin the ultrasound exposure time was increased up to about 70 ms, which caused damage of the upper layers of skin (in accordance with the results previously obtained using the pig model) (see FIG. 20).

The treatments of the pig and human skin show that the focused ultrasound device is able to generate coagulation lesions at a depth while leaving the upper skin layers intact. Our experiments show that the tissue layers lying deeper than the focal area also remain intact. Acoustic exposure times to produce such lesions were experimentally determined at different positions of the ultrasound focal area beneath the skin surface. Experiments with in-vitro porcine skin and NBTC stain technique verified that the disclosed device can create such lesions in a reproducible manner and the coagulated lesions lie from about 1 mm to about 2 mm below the skin surface at the specified settings, the coagulated skin is about 2 mm to about 3 mm long and about 1 mm in diameter. Additional experiments demonstrate that this approach to creating coagulation lesions works in ex-vivo human skin and acoustic parameters for coagulated lesion generation in ex-vivo human skin are close to the parameters used in the pig skin model.

### Experiment 4. Fractional Treatment

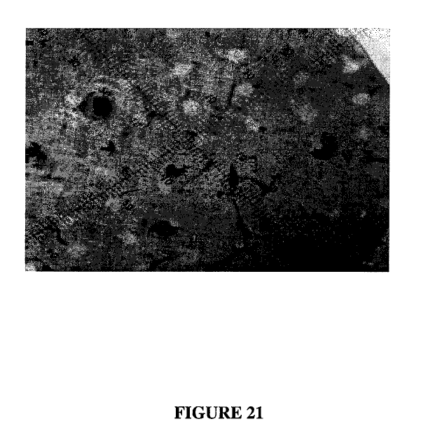

Thermo-coagulated fractions (e.g., islets) may be formed in skin using focused ultrasound. Using ultrasound, a matrix of lesions, with few micrometers or millimeters separation between, may be positioned in the dermis of skin (FIG. 21).

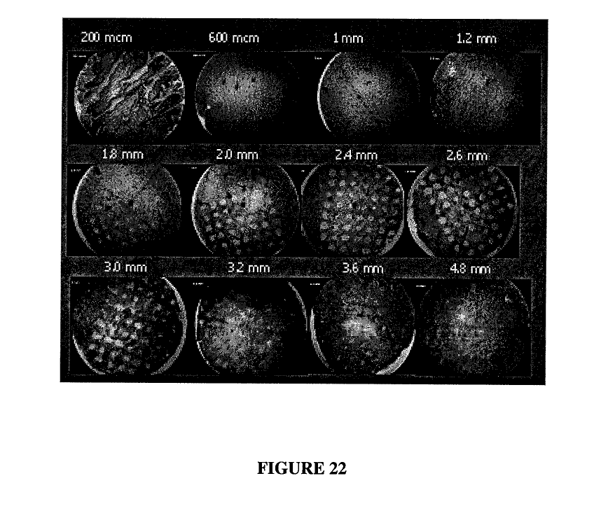

With reference to FIG. 22, slices of the pig skin (sliced medially) were treated by exposure to focused 5 MHz ultrasound. Each location was exposed to the focused ultrasound energy for about 10 ms. The focal depth was about 2.5 mm, the acoustical power density was from about 700 W/cm² to about 800 W/cm², and the F-number was about 1.0. The numbers above each slide indicate the slice's depth relative to the surface of the skin. Slices were cut using a cryotome with orientation of the blade parallel to the surface of skin and stained with NBTC. White circles are the coagulated spots in the dermis of skin, the average distance between the coagulated spots being about 1 mm. FIG. 22 shows that the coagulated region runs in the z direction from about 1.8 mm to about 3.2 or about 3.6 mm.

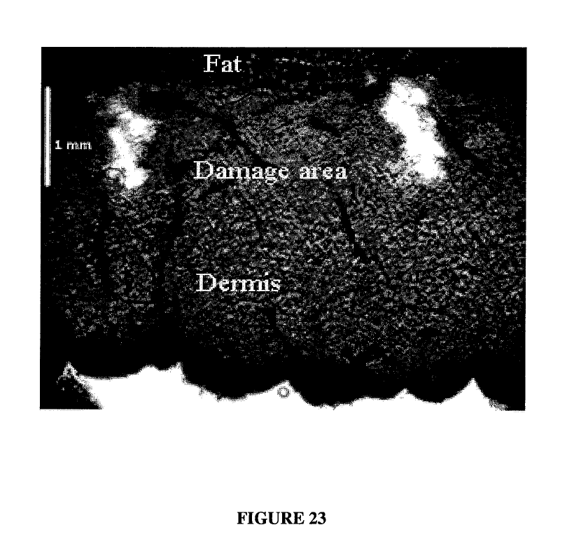

With reference to FIG. 23, two locations of a slice of the swine skin (sliced along the sagittal plane) were treated via exposure to focused 5 MHz ultrasound. Each location was exposed to the ultrasound energy for about 10 ms. The focal depth was about 2.5 mm, the acoustical power density was from about 700 W/cm² to about 800 W/cm², and the F-number was 1.0. Slices were cut using a cryotome with orientation of the blade perpendicular to the surface of the skin and stained with NBTC. White areas inside the dermis are the coagulated spots. Each coagulated region runs about 1 mm in the z direction.

One skilled in the art will appreciate further features and advantages of the disclosure based on the above-described embodiments. Accordingly, the disclosure is not to be limited by what has been particularly shown and described, except as indicated by the appended claims. All publications and references cited herein are expressly incorporated herein by reference in their entirety.

What is claimed is:

1. A method for treating tissue, comprising:
applying to a region of tissue one or more heating acoustic pulse with a frequency of from about 0.7 MHz to about 20 MHz and with a power density and an energy density sufficient to raise the temperature in the region of tissue at least about 5° C.; and
applying to the region of tissue one or more cavitation acoustic pulse with a frequency range of from about 20 kHz to about 700 kHz with a power density and an energy density sufficient to induce cavitation in the region of tissue.

2. The method of claim 1 wherein the heating acoustic pulse has a power density of from about 500 W/cm² to about 5,000 W/cm² and has an energy density of from about 2.5 J/cm² to about 25 J/cm².

3. The method of claim 1 wherein the cavitation acoustic pulse has a power density of from about 40 W/cm² to about 800 W/cm² and has an energy density of from about 4 J/cm² to about 80 J/cm².

4. The method of claim 1 wherein the one or more heating acoustic pulse has a frequency, a power density and an energy density sufficient to raise the temperature in the region of tissue from about 5° C. to about 35° C.

5. A device for applying ultrasound energy to tissue, comprising:
an ultrasound transducer for generating ultrasound energy for application to tissue; and
a mechanism for dispensing at least one acoustic coupling medium between the ultrasound transducer and a tissue portion to provide a substantially constant acoustic coupling of the ultrasound energy into said tissue portion during treatment.

6. The device of claim 5, wherein the mechanism is configured to continuously replenish the coupling medium during treatment.

7. The device of claim 5, wherein the coupling medium is selected such that the speed of sound through the coupling medium is substantially the same as the speed of sound through the tissue.

8. The device of claim 5, wherein the ultrasound transducer is disposed within a housing having a distal tip, the tip being sized and configured to dispense a desired amount of said at least one coupling medium onto a surface of said tissue portion.

9. The device of claim 8, wherein said at least one acoustic coupling medium comprises a first coupling medium disposed within the housing.

10. The device of claim 9, further comprising a reservoir containing a second coupling medium, the reservoir being in communication with the tip to transfer said second coupling medium to the tip to be dispensed onto said surface of the tissue portion.

11. The device of claim 10, wherein the first coupling medium and the second coupling medium are the same.

12. The device of claim 10, wherein the first coupling medium is a substance with an acoustic impedance substantially similar to an acoustic impedance of water.

13. The device of claim 10, wherein the first coupling medium has a viscosity of up to about 500,000 cPs.

14. The device of claim 10, further comprising a drive mechanism in communication with the reservoir, the drive mechanism configured to drive a desired amount of the second coupling medium from the reservoir to the tissue surface.

15. The device of claim 14, wherein the drive mechanism and a viscosity of the second coupling medium are configured to dispense a desired amount of the second coupling medium at a desired rate so as to provide a thin film of the second coupling medium between the tissue surface and tip.

16. The device of claim 15, wherein the drive mechanism is a piston.

17. The device of claim 15, wherein the thin film has a viscosity in a range of about 80,000 cPs to about 100,000 cPs.

18. The device of claim 15, wherein the thin film reduces friction between the tip and the tissue surface.

19. The device of claim 15, wherein the thin film provides cooling of the tissue surface.

20. The device of claim 15, wherein the thin film includes an additive configure to provide an indication of prior treatment.

21. The device of claim 20, wherein the additive is configured to change color if subjected to a predetermined amount of ultrasound energy.

22. The device of claim 5, wherein the mechanism is configured to dispense the coupling medium so as to substantially eliminate imperfections in the acoustic coupling medium during treatment.

23. A device, comprising:
a handheld housing;
a source for generating ultrasound energy for application to tissue through a distal end of the housing,
a reservoir for containing an acoustic coupling medium, said reservoir having an opening in proximity of said distal tip for dispensing said acoustic coupling medium onto a surface of the tissue to facilitate coupling of the ultrasound energy into said tissue portion, and
a drive mechanism coupled to said reservoir for driving said acoustic coupling medium from the reservoir onto said tissue surface.

24. The device of claim 23, wherein said drive mechanism and a viscosity of said acoustic medium are configured to provide a thin film of said acoustic medium onto the tissue surface as the housing is moved over the surface to apply ultrasound energy to said tissue.

25. A method of applying ultrasound energy to tissue, comprising:
providing a device having an ultrasound energy emitter and a reservoir for containing an acoustic coupling medium, said reservoir having an opening for dispensing said medium onto a tissue surface,
moving said device over a tissue surface while dispensing said coupling medium from the reservoir so as to form a thin film of the coupling medium on the tissue surface, and
activating said emitter to apply ultrasound energy to the tissue surface having said film of the coupling medium,
wherein said film of the coupling medium facilitates coupling of the ultrasound energy onto the tissue.

26. The method of claim 25, wherein said film has a substantially uniform thickness thereby to provide a substantially constant coupling between the ultrasound energy and different portions of the tissue.

27. The method of claim 25, wherein said steps of dispensing the coupling medium and applying the ultrasound energy are performed substantially concurrently.
